(19) United States
(12) Patent Application Publication
(10) Pub. No.: US 2021/0267983 A1
(43) Pub. Date: Sep. 2, 2021

(54) COMBINATIONS OF PPAR AGONISTS AND P38 KINASE INHIBITORS FOR PREVENTING OR TREATING FIBROTIC DISEASES

(71) Applicant: KINARUS AG, Basel (CH)

(72) Inventors: Alexander BAUSCH, Basel (CH); Matthew WRIGHT, (CH)

(21) Appl. No.: 17/259,828

(22) PCT Filed: Jul. 5, 2019
(86) PCT No.: PCT/EP2019/068079

§ 371 Date: Jan. 12, 2021

(30) Foreign Application Priority Data
Jul. 13, 2018 (EP) 18183317.9

(51) Int. Cl. A61K 31/519; A61K 31/4439; A61K 31/427; A61K 31/216; A61P 19/04; A61P 11/00

(52) U.S. Cl. CPC A61K 31/519; A61K 31/4439; A61P 11/00; A61K 31/216; A61P 19/04; A61K 31/427

(57) ABSTRACT

The present invention relates to a pharmaceutical combination comprising: (a) a PPAR agonist; (b) a p38 kinase inhibitor; and optionally (c) one or more pharmaceutically acceptable diluents, excipients or carriers for use in a method of preventing or treating fibrotic diseases or disorders in a subject.

### THE FIELD OF THE INVENTION

The present invention relates to methods of preventing or treating fibrotic diseases or disorders.

### BACKGROUND OF THE INVENTION

Fibrotic disorders are often devastating diseases leading to loss of specific organ function and poor quality of life. In the United States, the prevalence of fibroproliferative diseases is increasing due to aging of the population and the increase in obesity and metabolic disorders. Approximately 45% of deaths in the U.S. have been associated with the presence of one or more fibroproliferative disorders.

Fibrosis involves an excess accumulation of extracellular matrix (primarily composed of collagen) and usually results in loss of function when normal tissue is replaced with acellular scar tissue. Fibrotic diseases often affect specific organs. Fibrotic diseases of the liver include liver cirrhosis, which can result from chronic hepatitis B or C infection, diabetes, obesity, autoimmune injury, and chronic exposure to toxins including alcohol. Fibrotic diseases of the kidney are a consequence of kidney injury and can contribute to renal failure. The presence of tubulointerstitial and glomerular fibrosis in the kidney reflects chronic, progressive renal disease and is considered the primary mechanism leading to end-stage renal disease. The presence of Type I and II diabetes mellitus commonly underlie the development of renal fibrosis. Chronic renal insufficiency is often a sign of renal fibrosis and can be caused by exposure to chemicals and nephrotoxic drugs. In addition, genetic causes and infection can cause renal fibrosis. For example, fibrosis and loss of function of heart tissue is caused by hypoxic and ischemic events that reduce blood flow and tissue oxygenation. Cardiac myocytes may die and be replaced by fibrotic tissue.

For example, fibrotic diseases of the lung include interstitial lung disease, characterized by pulmonary inflammation and fibrosis. Such diseases of the lung may have specific causes including underlying vascular disease, sarcoidosis, silicosis, or systemic scleroderma. In most cases, however, the cause is unknown. Idiopathic pulmonary fibrosis is a rare but devastating disorder with no known cause. IPF is detected by differential diagnosis where other causes, such as the ones noted, are excluded. Interstitial lung diseases (ILDs) are associated with lung dysfunction due to fibrosis. Idiopathic Pulmonary Fibrosis (IPF) is a type of ILD that affects about 170,000 people in Europe and 130,000 people in the United States. IPF is a fatal disease with median survival of 3-5 years from diagnosis. Less than 30% survive beyond 5-years. Until recently, no effective treatment options other than lung transplantation had been shown to be effective. The most common cause of death is respiratory failure due to the progressive loss of lung function.

Recently, Nintedanib, a receptor tyrosine kinase inhibitor, was approved by the FDA for treatment of IPF. However, it is associated with significant systemic adverse events, including GI adverse reactions, liver enzyme elevation, decreased appetite, headache, weight loss, and hypertension. Pirfenidone was also approved by FDA in 2014 for the treatment of IPF. The mechanism of action of pirfenidone in the treatment of IPF has not been established.

Pirfenidone must be administered at a high dose (801 mg) three times daily with meals. Pirfenidone also has significant side effects including nausea, rash, abdominal pain, upper respiratory tract infection, diarrhea, fatigue, headache, dyspepsia, dizziness, vomiting, anorexia, gastro-esophageal reflux disease, and sinusitis. More importantly, both nintedanib and pirfenidone are only partially effective, reducing the rate of decline in lung function by approximately 50%. Neither drug affects all cause mortality or improves quality of life of patients with IPF.

Therefore, there remains a need for effective antifibrotic approaches to treat fibrotic diseases and, in particular, to improve the efficacy and tolerability of treatments for lung fibrotic diseases including IPF.

### SUMMARY OF THE INVENTION

It has now unexpectedly been found that a pharmaceutical combination comprising a PPAR agonist, such as pioglitazone and a p38 inhibitor, e.g. a compound of formula I or II as defined herein below, such as pamapimod, is useful for preventing or treating fibrotic diseases or disorders, in particular lung fibrotic diseases, such as IPF. In a standard model established in IPF research, it was surprisingly found that treatment with the pharmaceutical combination of the invention provides a greater effect to reduce fibrosis of the lung than treatment with a PPAR agonist or a p38 inhibitor alone and provides improved efficacy and tolerability when delivered orally. Moreover, the pharmaceutical combination was unexpectedly found to synergistically regulate the expression of multiple inflammatory genes of the interleukin/interleukin receptor, TNF/TNF receptor, and C-C and C-X-C motif chemokine gene families, indicating potentially potent anti-inflammatory effects, not exhibited by either agent alone.

Accordingly, in a first aspect, the present invention provides a pharmaceutical combination comprising:

(a) a PPAR agonist;

(b) a p38 kinase inhibitor; and optionally

(c) one or more pharmaceutically acceptable diluents, excipients or carriers for use in a method of preventing or treating fibrotic diseases or disorders in a subject.

In a further aspect, the present invention provides a kit for use in a method of preventing or treating fibrotic diseases or disorders in a subject, comprising a pharmaceutical combination comprising:

(a) a PPAR agonist;

(b) a p38 kinase inhibitor; and optionally

(c) one or more pharmaceutically acceptable diluents, excipients or carriers; and instructions for using the kit.

### BRIEF DESCRIPTION OF THE FIGURES

FIG. 1 shows lung weights normalized to individual animal body weights. Bleomycin instillation led to a significant increase in normalized lung weight when compared to non-bleomycin instilled sham controls. Substantially lower normalized lung weights were observed in groups treated with 25 mg/kg pioglitazone or 100 mg/kg pamapimod either alone or in combination (Groups 3, 4, and 5 vs. group 2, p<0.05, t test). Notably the combination reduced normalized lung weights to a greater extent than the positive control pirfenidone.

FIG. 2 shows the effect on fibrosis score in each treatment group. Induction with 1.5 U/kg bleomycin followed by 200 μL vehicle daily (Group 2) resulted in substantial fibrosis and the highest group mean fibrotic index (4.8). Substantially less fibrosis, as indicated by lower group mean fibrotic indices, was present in groups treated with 25 mg/kg pioglitazone or 100 mg/kg pamapimod either alone or in combination (Groups 3, 4, and 5 vs. group 2, p<0.05, t test). The lowest group mean fibrotic index was in Group 5, indicating that the combination of pioglitazone and pamapimod is more effective to reduce fibrosis score than either agent alone. Notably the combination reduced normalized fibrosis score to a greater extent than the positive control pirfenidone.

### DETAILED DESCRIPTION OF THE INVENTION

For the purposes of interpreting this specification, the following definitions will apply and whenever appropriate, terms used in the singular will also include the plural and vice versa. It is to be understood that the terminology used herein is for the purpose of describing particular embodiments only and is not intended to be limiting.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any embodiment. The invention extends to any novel one, or any novel combination, of the features disclosed in this specification (including any accompanying claims, abstract and drawings), or to any novel one, or any novel combination, of the steps of any method or process so disclosed.

The terms “comprising”, “having”, and “including” are to be construed as open-ended terms (i.e., meaning “including, but not limited to”) unless otherwise noted.

The term “pharmaceutically acceptable diluents, excipients or carriers” as used herein refers to diluents, excipients or carriers that are suitable for use with humans and/or animals without undue adverse side effects (such as toxicity, irritation, and allergic response) commensurate with a reasonable benefit/risk ratio. “Diluents” are agents which are added to the bulk volume of the active agent making up the solid composition. As a result, the size of the solid composition increases, which makes it easier to handle. Diluents are convenient when the dose of drug per solid composition is low and the solid composition would otherwise be too small. “Excipients” can be binders, lubricants, glidants, coating additives or combinations thereof. Thus, excipients are intended to serve multiple purposes. “Carriers” can be solvents, suspending agents or vehicles, for delivering the instant compounds to a subject.

The term “fibrotic diseases or disorders” is intended to refer to medical conditions of the body known in the art related to diseases or disorders caused by organs which develop excess fibrous connective tissue with impaired function. Accordingly, the term “fibrotic diseases or disorders” is meant to include, but is not limited to, diseases or disorders selected from the group consisting of lung fibrosis, liver fibrosis, kidney fibrosis, cardiac fibrosis, ocular fibrosis or cutaneous fibrosis.

The term “lung fibrosis” refers to a group of fibrotic diseases or disorders affecting the lung, such as idiopathic pulmonary fibrosis (IPF), familial interstitial pulmonary fibrosis, nonspecific interstitial pneumonia (NSIP), cryptogenic organizing pneumonia (COP), sarcoidosis, chronic obstructive pulmonary disease (COPD), and asbestosis. “Fibrotic diseases or disorders affecting the lung” or “fibrotic lung disease or disorder” are terms which are used interchangeably herein and refer to a respiratory disease in which scars are formed due to the accumulation of excess fibrous connective tissue in the lung tissues, mainly collagen, leading to progressive loss of lung function. The accumulation of excess fibrous connective tissue leads to thickening of alveolar walls, leading to reduced lung function. As a consequence, patients suffer from shortness of breath, are unable to exert themselves physically, and are at risk for pneumonia or other serious lung infections. In some patients, the specific cause of the disease can be diagnosed, but in others the probable cause cannot be determined, a condition called idiopathic pulmonary fibrosis (IPF). There are no treatments to reverse fibrosis or prevent further loss of lung function. Recently, Nintedanib, a receptor tyrosine kinase inhibitor, was approved by the FDA for treatment of IPF. However, it is associated with significant systemic adverse events, including GI adverse reactions, liver enzyme elevation, decreased appetite, headache, weight loss, and hypertension. Pirfenidone was also approved by FDA in 2014 for the treatment of IPF. Pirfenidone also has significant side effects including nausea, rash, abdominal pain, upper respiratory tract infection, diarrhea, fatigue, headache, dyspepsia, dizziness, vomiting, anorexia, gastro-esophageal reflux disease, and sinusitis. More importantly, both nintedanib and pirfenidone are only partially effective, reducing the rate of decline in lung function by approximately 50%. Neither drug affects all cause mortality or improves quality of life of patients with IPF.

The term “idiopathic pulmonary fibrosis (IPF)” refers to a disease of the lung that occurs in middle-aged and elderly adults (median age at diagnosis 66 years, range 55-75 years). IPF is limited to the lungs, and is associated with a histopathological or radiological pattern typical of usual interstitial pneumonia. The cause of IPF is unknown, however a history of smoking, genetic factors, and environmental insults are thought to trigger the pathological changes that lead to fibrotic remodeling of lung tissue. After lung injury, epithelial cells release inflammatory mediators that initiate an anti-fibrinolytic coagulation cascade, which triggers platelet activation and blood clot formation. This is followed by entry of leukocytes (e.g., neutrophils, macrophages, and T cells). The recruited leukocytes secrete pro-fibrotic cytokines such as IL-1β, TNF-α, and TGF-β. In the subsequent phase, fibroblasts and myofibroblasts are derived from epithelial cells undergoing epithelial to mesenchymal transition, as well as fibrocytes from the bone marrow, and resident fibroblasts that proliferate and differentiate into myofibroblasts. These cells release collagen and other fibrotic components. IPF is a chronic, progressive, irreversible, and eventually lethal lung disease. Life expectancy after diagnosis ranges from 3-5 years.

The term “familial interstitial pulmonary fibrosis” refers to a disease similar to IPF, in which affects two or more related individuals. Familial interstitial pulmonary fibrosis has been suggested to be associated with changes in telomere length or gene mutations.

The term “ nonspecific interstitial pneumonia (NSIP)” refers to a disease caused by reactions to certain medications, HIV, as well as other conditions. The disease is characterized mainly by inflammation of the cells of the interstitium. The fibrotic form is defined by thickening and scarring of lung tissue.

The term “cryptogenic organizing pneumonia (COP)” refers to a disease with alveolar inflammatory changes similar to regular pneumonia, but also with involvement of the bronchioles. Histologically, COP is characterised by mild patchy interstitial inflammation without fibrosis, and the presence of buds of granulation tissue made of mononuclear cells, foamy macrophages, and fibrous tissue (Masson bodies) in distal airspaces.

The term “sarcoidosis” refers to a disease involving abnormal collections of inflammatory cells into lumps called granulomas. When it affects the lungs there may be wheezing, coughing, shortness of breath, or chest pain.

The term chronic obstructive pulmonary disease (COPD) refers to a chronic inflammatory lung disease that causes obstructed airflow from the lungs. Symptoms include breathing difficulty, cough, mucus (sputum) production and wheezing.

The term “asbestosis” refers to a disease characterized by long term inflammation and scarring of the lungs due to asbestos exposure.

The term “pharmaceutically acceptable salt” of a compound means a salt that is pharmaceutically acceptable and that it possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxy-benzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e. g. an alkaline metal ion, an alkaline earth metal ion, or an aluminum ion; or coordinates with an organic base such as ethanolamine, diethanolamine, triethanolamine, tromethamine, N-methylglucamine, and the like. Preferred salts comprise acid addition salts formed with hydrochloric acid.

The terms “subject” and “patient” are used herein interchangeably and refer to mammals, in particular humans.

The term “about” as used herein refers to +/−10% of a given measurement.

In a first aspect, the present invention provides a pharmaceutical combination comprising:

(a) a PPAR agonist;

(b) a p38 kinase inhibitor; and optionally

(c) one or more pharmaceutically acceptable diluents, excipients or carriers for use in a method of preventing or treating fibrotic diseases or disorders in a subject.

PPAR Agonists

The term “PPAR agonist ” as used herein refers to a drug that is activating peroxisome proliferator activated receptor (PPAR) such as PPAR gamma receptor, PPAR alpha receptor, PPAR delta receptor or combinations thereof and includes PPAR gamma agonists such as e.g. pioglitazone, troglitazone or rosiglitazone, PPAR alpha agonists such as e.g. fibrates such as bezafibrate, fenofibrate (fenofibric acid), clofibrate or gemfibrozil, PPAR dual agonists (PPAR alpha/gamma or PPAR alpha/delta agonists) such as e.g. aleglitazar, muraglitazar, tesaglitazar, ragaglitazar, saroglitazar, GFT505 or naveglitazar, PPAR delta agonists such as e.g. GW501516, PPAR pan agonists (PPAR alpha/delta/gamma agonists) or selective PPAR modulators such as e.g. INT131 and the pharmaceutically acceptable salts of these compounds. Usually PPAR gamma agonists, PPAR modulators, PPAR alpha agonists and/or PPAR alpha/gamma dual agonists are used in the pharmaceutical combinations of the present invention, in particular PPAR gamma agonists, PPAR alpha agonists and/or PPAR alpha/gamma dual agonists are used in the pharmaceutical combinations of the present invention, more particularly PPAR gamma agonists and/or PPAR alpha agonists selected from the group consisting of pioglitazone, rosiglitazone, troglitazone, fenofibrate, bezafibrate and pharmaceutically acceptable salts thereof, even more particularly PPAR gamma agonists selected from the group consisting of pioglitazone, rosiglitazone, troglitazone and pharmaceutically acceptable salts thereof, preferably pioglitazone or pharmaceutically acceptable salts thereof. PPAR alpha agonists used in the pharmaceutical combinations of the present invention are selected from the group consisting of bezafibrate, fenofibrate (fenofibric acid), clofibrate, gemfibrozil and pharmaceutically acceptable salts thereof, preferably bezafibrate, fenofibrate (fenofibric acid) or pharmaceutically acceptable salts thereof, more preferably bezafibrate or pharmaceutically acceptable salts thereof. PPAR alpha/gamma dual agonists used in the pharmaceutical combinations of the present invention are selected from the group consisting of aleglitazar, muraglitazar, tesaglitazar, ragaglitazar, saroglitazar, GFT505, naveglitazar and pharmaceutically acceptable salts thereof, preferably muraglitazar, tesaglitazar or pharmaceutically acceptable salts thereof. Preferably PPAR gamma agonists and/or PPAR alpha agonists are used in the pharmaceutical combinations of the present invention, more preferably PPAR gamma agonists or modulators and/or PPAR alpha agonists selected from the group consisting of pioglitazone, rosiglitazone, troglitazone, fenofibrate, bezafibrate, INT131 and pharmaceutically acceptable salts thereof, even more preferably PPAR gamma agonists selected from the group consisting of pioglitazone, rosiglitazone, troglitazone and pharmaceutically acceptable salts thereof are used. Even more preferably, pioglitazone or a pharmaceutically acceptable salt thereof, in particular pioglitazone hydrochloride is used in the pharmaceutical combinations of the present invention. In one embodiment, a thiazolidinedione PPAR agonist is used in the pharmaceutical combinations of the invention. Suitable thiazolidinedione PPAR agonists are for example pioglitazone, troglitazone, rosiglitazone or pharmaceutically acceptable salts thereof. A particularly suitable thiazolidinone PPAR agonist is pioglitazone or a pharmaceutically acceptable salt thereof, in particular pioglitazone hydrochloride.

Pioglitazone is described e.g. in U.S. Pat. No. 4,687,777 or in Dormandy J A, Charbonnel B, Eckland D J, Erdmann E, Massi-Benedetti M, Moules I K, Skene A M, Tan M H, Lefebvre P J, Murray G D, Standl E, Wilcox R G, Wilhelmsen L, Betteridge J, Birkeland K, Golay A, Heine R J, Korányi L, Laakso M, Mokán M, Norkus A, Pirags V, Podar T, Scheen A, Scherbaum W, Schernthaner G, Schmitz O, Skrha J, Smith U, Taton J; PROactive investigators. Lancet. 2005 Oct 8; 366(9493):1279-89, and is represented by the structural formula indicated below:

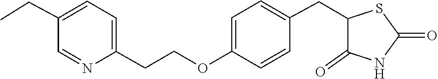 [SMILES: CCc1ccc(CCOc2ccc(CC3SC(=O)NC3=O)cc2)nc1]

Troglitazone is described e.g. in Florez J C, Jablonski K A, Sun M W, Bayley N, Kahn S E, Shamoon H, Hamman R F, Knowler W C, Nathan D M, Altshuler D; Diabetes Prevention Program Research Group. J Clin Endocrinol Metab. 2007 Apr; 92(4):1502-9 and is represented by the structural formula indicated below:

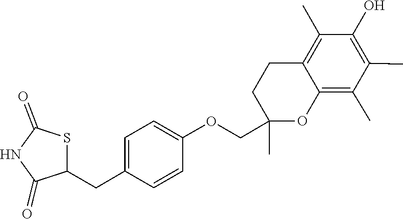 [SMILES: Cc1c(C)c2c(c(C)c1O)CCC(C)(COc1ccc(CC3SC(=O)NC3=O)cc1)O2]

Rosiglitazone is described e.g. in Nissen S E, Wolski K. N Engl J Med. 2007 Jun 14; 356(24):2457-71. Erratum in: N Engl J Med. 2007 Jul 5;357(1):100. Fenofibrate is described e.g. in Bonds D E, Craven T E, Buse J, Crouse J R, Cuddihy R, Elam M, Ginsberg H N, Kirchner K, Marcovina S, Mychaleckyj J C, O'Connor P J, Sperl-Hillen J A. Diabetologia. 2012 Jun; 55(6):1641-50 and is represented by the structural formula indicated below:

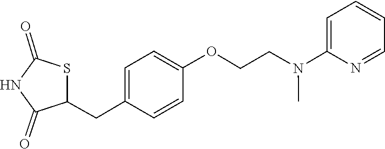 [SMILES: CN(CCOc1ccc(CC2SC(=O)NC2=O)cc1)c1ccccn1]

Bezafibrate is described e.g. in I. Goldenberg, M. Benderly, U. Goldbourt, Vascular health and risk management. 2008, 4(1): 131-141 and is represented by the structural formula indicated below:

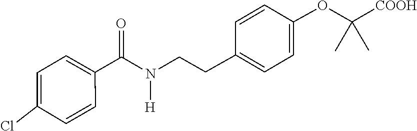 [SMILES: CC(C)(Oc1ccc(CCNC(=O)c2ccc(Cl)cc2)cc1)C(=O)O]

Clofibrate is described e.g. in Rabkin S W, Hayden M, Frohlich J. Atherosclerosis. 1988 Oct; 73(2-3):233-40 and is represented by the structural formula indicated below:

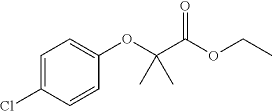 [SMILES: CCOC(=O)C(C)(C)Oc1ccc(Cl)cc1]

Fenofibrate (fenofibric acid) is described e.g. in Schima S M, Maciejewski S R, Hilleman D E, Williams M A, Mohiuddin S M. Expert Opin Pharmacother. 2010 Apr; 11(5):731-8 and is represented by the structural formula indicated below:

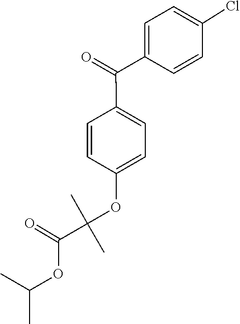 [SMILES: CC(C)OC(=O)C(C)(C)Oc1ccc(C(=O)c2ccc(Cl)cc2)cc1]

Gemfibrozil is described e.g. in Adabag A S, Mithani S, Al Aloul B, Collins D, Bertog S, Bloomfield H E; Veterans Affairs High-Density Lipoprotein Cholesterol Intervention Trial Study Group. Am Heart J. 2009 May; 157(5):913-8 and is represented by the structural formula indicated below:

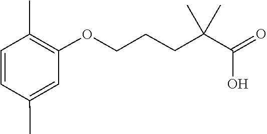 [SMILES: Cc1ccc(C)c(OCCCC(C)(C)C(=O)O)c1]

Aleglitazar is described e.g. in Lincoff A M, Tardif J C, Schwartz G G, Nicholls S J, Ryden L, Neal B, Malmberg K, Wedel H, Buse J B, Henry R R, Weichert A, Cannata R, Svensson A, Volz D, Grobbee D E; AleCardio Investigators. JAMA. 2014 Apr 16; 311(15):1515-25 and is represented by the structural formula indicated below:

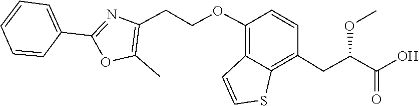 [SMILES: CO[C@@H](Cc1ccc(OCCc2nc(-c3ccccc3)oc2C)c2ccsc12)C(=O)O]

Muraglitazar is described e.g. in Fernandez M, Gastaldelli A, Triplitt C, Hardies J, Casolaro A, Petz R, Tantiwong P, Musi N, Cersosimo E, Ferrannini E, DeFronzo R A. Diabetes Obes Metab. 2011 Oct; 13(10):893-902 and is represented by the structural formula indicated below:

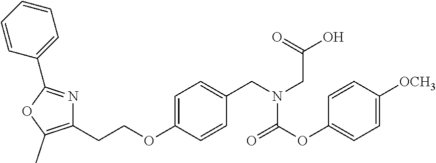 [SMILES: COc1ccc(OC(=O)N(CC(=O)O)Cc2ccc(OCCc3nc(-c4ccccc4)oc3C)cc2)cc1]

Tesaglitazar is described e.g. in Bays H, McElhattan J, Bryzinski BS; GALLANT 6 Study Group. Diab Vasc Dis Res. 2007 Sep; 4(3):181-93 and is represented by the structural formula indicated below:

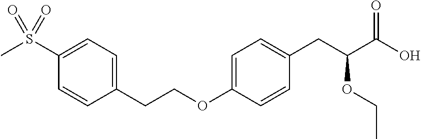 [SMILES: CCO[C@@H](Cc1ccc(OCCc2ccc(S(C)(=O)=O)cc2)cc1)C(=O)O]

Ragaglitazar is described e.g. in Saad M F, Greco S, Osei K, Lewin A J, Edwards C, Nunez M, Reinhardt R R; Ragaglitazar Dose-Ranging Study Group. Diabetes Care. 2004 Jun; 27(6):1324-9 and is represented by the structural formula indicated below:

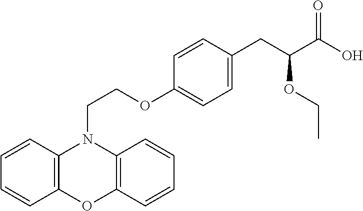 [SMILES: CCO[C@@H](Cc1ccc(OCCN2c3ccccc3Oc3ccccc32)cc1)C(=O)O]

Saroglitazar is described e.g. in Agrawal R. Curr Drug Targets. 2014 Feb; 15(2):151-5. and is represented by the structural formula indicated below:

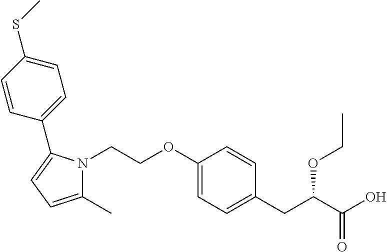 [SMILES: CCO[C@@H](Cc1ccc(OCCn2c(C)ccc2-c2ccc(SC)cc2)cc1)C(=O)O]

Naveglitazar is described e.g. in Ahlawat P, Srinivas N R. Eur J Drug Metab Pharmacokinet. 2008 Jul-Sep; 33(3):187-90. GW501516 is described e.g. in Wang X, Sng M K, Foo S, Chong H C, Lee W L, Tang M B, Ng K W, Luo B, Choong C, Wong M T, Tong B M, Chiba S, Loo S C, Zhu P, Tan N S. J Control Release. 2015 Jan 10; 197:138-47 and is represented by the structural formula indicated below:

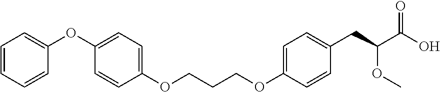 [SMILES: CO[C@@H](Cc1ccc(OCCCOc2ccc(Oc3ccccc3)cc2)cc1)C(=O)O]

GFT505 is described e.g. in Cariou B, Staels B. Expert Opin Investig Drugs. 2014 Oct; 23(10):1441-8 and is represented by the structural formula indicated below:

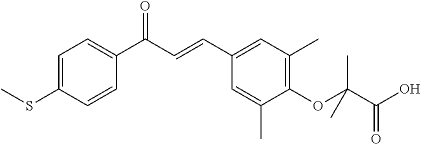 [SMILES: CSc1ccc(C(=O)/C=C/c2cc(C)c(OC(C)(C)C(=O)O)c(C)c2)cc1]

INT131 is described e.g. in. Taygerly J P, McGee L R, Rubenstein S M, Houze J B, Cushing T D, Li Y, Motani A, Chen J L, Frankmoelle W, Ye G, Learned M R, Jaen J, Miao S, Timmermans P B, Thoolen M, Kearney P, Flygare J, Beckmann H, Weiszmann J, Lindstrom M, Walker N, Liu J, Biermann D, Wang Z, Hagiwara A, Iida T, Aramaki H, Kitao Y, Shinkai H, Furukawa N, Nishiu J, Nakamura M. Bioorg Med Chem. 2013 Feb 15; 21(4):979-92 and is represented by the structural formula indicated below:

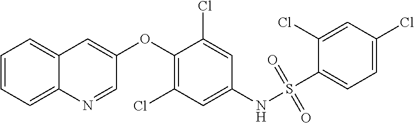 [SMILES: O=S(=O)(Nc1cc(Cl)c(Oc2cnc3ccccc3c2)c(Cl)c1)c1ccc(Cl)cc1Cl]

PPAR activation by the PPAR agonist is usually strong in the low nanomolar range to low micromolar range, e.g in a range of 0.1 nM to 100 μM. In some embodiments the PPAR activation is weak or partial, i.e. a PPAR agonist is used in the methods of the present invention which yields maximal activation of PPAR-receptor in a reporter assay system of 10% to 100% compared to a reference PPAR agonist which is known to causes a maximum PPAR activation.

p38 Kinase Inhibitors

The term “p38 kinase inhibitor” or “p38 inhibitor” which are both used interchangeably herein refers to a drug that is inhibiting a p38 mitogen-activated protein (MAP) kinase, such as p38-alpha (MAPK14), p38-beta (MAPK11), p38-gamma (MAPK12/ERK6), and/or p38-delta (MAPK13/SAPK4). Examples of p38 inhibitors include compounds of formulae I and II and pharmaceutically acceptable salts thereof as defined herein. Further examples of p38 inhibitors include pamapimod, acumapimod, losmapimod, dilmapimod, semapimod, AZD7624, ARRY-371797, LY2228820, R9111, PH-797804, BIRB 796, VX-702, VX-745, SB 239063, SB202190, SCIO 469, BMS 582949 and pharmaceutically acceptable salts thereof.

In one embodiment, the pharmaceutical combination according to the invention comprises:

(a) a PPAR agonist;

(b) a p38 kinase inhibitor; and optionally

(c) one or more pharmaceutically acceptable diluents, excipients or carriers;

wherein said p38 inhibitor is inhibiting p38-alpha, p38-beta, p38-gamma or p38-delta or combinations thereof, preferably inhibiting p38-alpha and/or p38-beta, more preferably inhibiting p38-alpha.

In a further embodiment, the p38 inhibitor for use in a pharmaceutical combination according to the invention is a compound of formula I or II

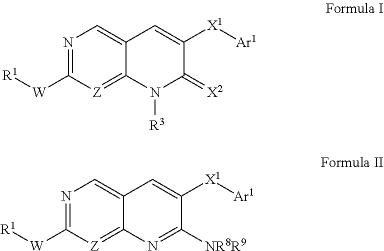

or a pharmaceutically acceptable salt thereof, wherein

Z is N or CH;

W is NR²;

X¹ is O, NR⁴ (where R⁴ is hydrogen or alkyl), S, or CR⁵R⁶ (where R⁵ and R⁶ are independently hydrogen or alkyl) or C═O;

X² is O or NR⁷;

Ar¹ is aryl or heteroaryl;

R² is hydrogen, alkyl, acyl, alkoxycarbonyl, aryloxycarbonyl, heteroalkylcarbonyl, heteroalkyloxycarbonyl or —R²¹—R²² where R²¹ is alkylene or —C(═O)— and R²² is alkyl or alkoxy;

R¹ is hydrogen, alkyl, haloalkyl, aryl, aralkyl, heteroaryl, heteroaralkyl, cycloalkyl, cycloalkylalkyl, heteroalkylsubstituted cycloalkyl, heterosubstituted cycloalkyl, heteroalkyl, cyanoalkyl, heterocyclyl, heterocyclylalkyl, R¹²—SO₂-heterocycloamino (where R¹² is haloalkyl, aryl, aralkyl, heteroaryl or heteroaralkyl), —Y¹—C(O)—Y²—R¹¹ (where Y¹ and Y² are independently either absent or an alkylene group and R¹¹ is hydrogen, alkyl, halo alkyl, hydroxy, alkoxy, amino, monoalkylamino or dialkylamino), (heterocyclyl)(cycloalkyl)alkyl or (heterocyclyl)(heteroaryl)alkyl;

R³ is hydrogen, alkyl, cycloalkyl, cycloalkylalkyl, aryl, aralkyl, haloalkyl, heteroalkyl, cyanoalkyl, alkylene-C(O)—R³¹ (where R³¹ is hydrogen, alkyl, hydroxy, alkoxy, amino, monoalkylamino or dialkylamino), amino, monoalkylamino, dialkylamino or NR³²—Y³—R³³ (where Y³ is —C(O), —C(O)O—, —C(O)NR³⁴, S(O)₂ or S(O)₂NR³⁵; R³², R³⁴ and R³⁵ are independently hydrogen or alkyl; and R³³ is hydrogen, alkyl, cycloalkyl, cycloalkylalkyl, heteroalkyl or optionally substituted phenyl) or acyl;

R⁷ is hydrogen or alkyl; and

R⁸ and R⁹ are independently hydrogen, alkyl, aryl, aralkyl, cycloalkyl, cycloalkylalkyl, heteroalkyl, alkylsulfonyl, arylsulfonyl, —C(O)—R⁸¹ (where R⁸¹ is alkyl, aryl, aralkyl, cycloalkyl, cycloalkylalkyl, heteroalkyl, alkoxy, aryloxy, amino, mono- or di-alkylamino, arylamino or aryl(alkyl)amino) or R⁸ and R⁹ together form ═CR⁸²R⁸³ (where R⁸² and R⁸³ are independently hydrogen, alkyl, cycloalkyl, cycloalkylalkyl or optionally substituted phenyl) and optionally one or more pharmaceutically acceptable diluents, excipients or carriers.

In a preferred embodiment, the p38 inhibitor for use in a pharmaceutical combination according to the invention is a compound of formula I

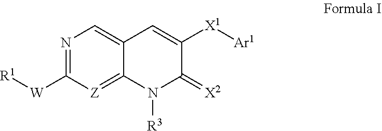

or a pharmaceutically acceptable salt thereof, wherein

Z is N or CH;

W is NR²;

X¹ is O, NR⁴ (where R⁴ is hydrogen or alkyl), S, or CR⁵R⁶ (where R⁵ and R⁶ are independently hydrogen or alkyl) or C═O;

X² is O or NR⁷;

Ar¹ is aryl or heteroaryl;

R² is hydrogen, alkyl, acyl, alkoxycarbonyl, aryloxycarbonyl, heteroalkylcarbonyl, heteroalkyloxycarbonyl or —R²¹—R²² where R²¹ is alkylene or —C(═O)— and R²² is alkyl or alkoxy;

R¹ is hydrogen, alkyl, haloalkyl, aryl, aralkyl, heteroaryl, heteroaralkyl, cycloalkyl, cycloalkylalkyl, heteroalkylsubstituted cycloalkyl, heterosubstituted cycloalkyl, heteroalkyl, cyanoalkyl, heterocyclyl, heterocyclylalkyl, R¹²—SO₂-heterocycloamino (where R¹² is haloalkyl, aryl, aralkyl, heteroaryl or heteroaralkyl), —Y¹—C(O)—Y²—R¹¹ (where Y¹ and Y² are independently either absent or an alkylene group and R¹¹ is hydrogen, alkyl, halo alkyl, hydroxy, alkoxy, amino, monoalkylamino or dialkylamino), (heterocyclyl)(cycloalkyl)alkyl or (heterocyclyl)(heteroaryl)alkyl;

R³ is hydrogen, alkyl, cycloalkyl, cycloalkylalkyl, aryl, aralkyl, haloalkyl, heteroalkyl, cyanoalkyl, alkylene-C(O)—R³¹ (where R³¹ is hydrogen, alkyl, hydroxy, alkoxy, amino, monoalkylamino or dialkylamino), amino, monoalkylamino, dialkylamino or NR³²—Y³—R³³ (where Y³ is —C(O), —C(O)O—, —C(O)NR³⁴, S(O)₂ or S(O)₂NR³⁵; R³², R³⁴ and R³⁵ are independently hydrogen or alkyl; and R³³ is hydrogen, alkyl, cycloalkyl, cycloalkylalkyl, heteroalkyl or optionally substituted phenyl) or acyl; and

R⁷ is hydrogen or alkyl and optionally one or more pharmaceutically acceptable diluents, excipients or carriers.

In a further embodiment, the p38 inhibitor for use in a pharmaceutical combination according to the invention is a compound of formula I wherein X¹ is NR⁴ and X² is NR⁷ or X¹ and X² are each O, wherein R⁴ and R⁷ are as defined above.

In a further embodiment, the p38 inhibitor for use in a pharmaceutical combination according to the invention is a compound of formula I wherein X¹ is NR⁴ or O and X² is NR⁷ or O, wherein R⁴ and R⁷ are as defined above.

In a further embodiment, the p38 inhibitor for use in a pharmaceutical combination according to the invention is a compound of formula I wherein W is NR² and wherein R² is hydrogen, alkyl, heteroalkyl, acyl or alkoxycarbonyl, preferably hydrogen or alkyl, more preferably hydrogen.

In yet a further embodiment, the p38 inhibitor for use in a pharmaceutical combination according to the invention is a compound of formula I wherein R¹ is hydrogen, alkyl, haloalkyl, cycloalkyl, cycloalkylalkyl, heteroalkylsubstituted cycloalkyl, heterosubstituted cycloalkyl, heteroalkyl, cyanoalkyl, heterocyclyl, heterocyclylalkyl or (heterocyclyl)(cycloalkyl)alkyl.

In a preferred embodiment, the p38 inhibitor for use in a pharmaceutical combination according to the invention is a compound of formula I wherein R² is hydrogen and R¹ is heteroalkyl or vice versa.

In yet a further embodiment, the p38 inhibitor for use in a pharmaceutical combination according to the invention is a compound of formula I wherein R¹ is hydrogen, alkyl, haloalkyl, heteroalkyl or cyanoalkyl.

In yet a further embodiment, the p38 inhibitor for use in a pharmaceutical combination according to the invention is a compound of formula I wherein R¹ is cycloalkyl, cycloalkylalkyl, heteroalkylsubstituted cycloalkyl, heterosubstituted cycloalkyl, heterocyclyl, heterocyclylalkyl or (heterocyclyl)(cycloalkyl)alkyl.

In a preferred embodiment, the p38 inhibitor for use in a pharmaceutical combination according to the invention is a compound of formula I wherein each of R¹ and R² is independently selected from hydrogen and hydroxyalkyl, preferably from hydrogen, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 2-hydroxy-1-hydroxymethylethyl, 2,3-dihydroxybutyl, 3,4-dihydroxybutyl, 2-(hydroxymethyl)-3-hydroxypropyl, 3-hydroxy-1-(2-hydroxyethyl)-propyl and 2-hydroxy-1-methylethyl, more preferably from hydrogen, 2-hydroxyethyl, 2,3-dihydroxypropyl and 1-(hydroxymethyl)2-hydroxyethyl, most preferably from hydrogen, 2-hydroxy-propyl, 3-hydroxy-1-(2-hydroxyethyl)-propyl and 2-hydroxy-1-methylethyl.

In yet a further embodiment, the p38 inhibitor for use in a pharmaceutical combination according to the invention is a compound of formula I wherein R³ is hydrogen, alkyl, cycloalkyl, cycloalkylalkyl, haloalkyl, heteroalkyl, cyanoalkyl, alkylene-C(O)—R³¹ (where R³¹ is hydrogen, alkyl, hydroxy, alkoxy, amino, monoalkylamino or dialkylamino) or acyl.

In yet a further embodiment, the p38 inhibitor for use in a pharmaceutical combination according to the invention is a compound of formula I wherein R³ is hydrogen, alkyl, haloalkyl, heteroalkyl, cyanoalkyl, cycloalkyl or cycloalkylalkyl.

In yet a further embodiment, the p38 inhibitor for use in a pharmaceutical combination according to the invention is a compound of formula I wherein R³ is hydrogen, alkyl, haloalkyl, heteroalkyl or cyanoalkyl.

In yet a further embodiment, the p38 inhibitor for use in a pharmaceutical combination according to the invention is a compound of formula I wherein R³ is cycloalkyl or cycloalkylalkyl.

In yet a further embodiment, the p38 inhibitor for use in a pharmaceutical combination according to the invention is a compound of formula I wherein X¹ and X² are both O.

In yet a further embodiment, the p38 inhibitor for use in a pharmaceutical combination according to the invention is a compound of formula I wherein R¹ is alkyl or heteroalkyl.

In yet a further embodiment, the p38 inhibitor for use in a pharmaceutical combination according to the invention is a compound of formula I wherein R¹ is heteroalkyl, preferably 3-hydroxy-1-(2-hydroxyethyl)-propyl or 2-hydroxy-1-methylethyl.

In yet a further embodiment, the p38 inhibitor for use in a pharmaceutical combination according to the invention is a compound of formula I wherein R³ is alkyl or heteroalkyl.

In yet a further embodiment, the p38 inhibitor for use in a pharmaceutical combination according to the invention is a compound of formula I wherein R³ is alkyl, preferably C1-C5 alkyl, more preferably C1-C4 alkyl, more preferably C1-C3 alkyl. In a particularly preferred embodiment, R³ is ethyl or methyl, preferably methyl.

In yet a further embodiment, the p38 inhibitor for use in a pharmaceutical combination according to the invention is a compound of formula I wherein R³ is heteroalkyl, preferably 2-hydroxy-propyl.

In yet a further embodiment, the p38 inhibitor for use in a pharmaceutical combination according to the invention is a compound of formula I wherein W is NH.

In yet a further embodiment, the p38 inhibitor for use in a pharmaceutical combination according to the invention is a compound of formula I wherein Z is N.

In yet a further embodiment, the p38 inhibitor for use in a pharmaceutical combination according to the invention is a compound of formula I wherein Ar¹ is aryl, preferably phenyl optionally substituted with one, two or three halo substituents, most preferably phenyl substituted with two halo substituents in ortho and para position. In a particularly preferrred embodiment, Ar¹ is 2,4-difluorophenyl.

In yet a further embodiment, the p38 inhibitor for use in a pharmaceutical combination according to the invention is a compound of formula I wherein X¹ is NR⁴ and X² is NR⁷ or X¹ and X² are each O, wherein R⁴ and R⁷ are as defined above; and wherein

R¹ is hydrogen, alkyl, haloalkyl, cycloalkyl, cycloalkylalkyl, heteroalkylsubstituted cycloalkyl, heterosubstituted cycloalkyl, heteroalkyl, cyanoalkyl, heterocyclyl, heterocyclylalkyl or (heterocyclyl)(cycloalkyl)alkyl; and wherein

R³ is hydrogen, alkyl, cycloalkyl, cycloalkylalkyl, haloalkyl, heteroalkyl, cyanoalkyl, alkylene-C(O)—R³¹ (where R³¹ is hydrogen, alkyl, hydroxy, alkoxy, amino, monoalkylamino or dialkylamino) or acyl; and wherein

W is NR², wherein R² is hydrogen, alkyl, acyl or alkoxycarbonyl; and wherein

Ar¹ is aryl; and wherein

Z is N.

In a preferred embodiment, the p38 inhibitor for use in a pharmaceutical combination according to the invention is a compound of formula I wherein X¹ and X² are each O and wherein Z is N and wherein W is NH and wherein Ar¹ is phenyl optionally substituted by one, two or three halo substituents and wherein R¹ is heteroalkyl and wherein R³ is alkyl or heteroalkyl.

In a further embodiment, the p38 inhibitor for use in a pharmaceutical combination according to the invention is a compound of formula II

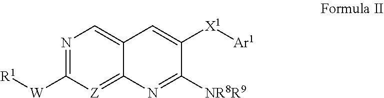

or a pharmaceutically acceptable salt thereof, wherein Ar¹, W, X¹, Z, R¹, R⁸ and R⁹ are as defined in any of the embodiments above.

Unless otherwise stated, the following terms have the meanings given below: “Acyl” means a radical —C(O)R, where R is hydrogen, alkyl, cycloalkyl, cycloalkylalkyl, phenyl or phenylalkyl wherein alkyl, cycloalkyl, cycloalkylalkyl, and phenylalkyl are as defined herein. Representative examples include, but are not limited to formyl, acetyl, cylcohexylcarbonyl, cyclohexylmethylcarbonyl, benzoyl, benzylcarbonyl, and the like. “Acylamino” means a radical-NR′C(O)R, where R¹ is hydrogen or alkyl, and R is hydrogen, alkyl, cycloalkyl, cycloalkylalkyl, phenyl or phenylalkyl wherein alkyl, cycloalkyl, cycloalkylalkyl, and phenylalkyl are as defined herein. Representative examples include, but are not limited to formylamino, acetylamino, cylcohexylcarbonylamino, cyclohexylmethyl-carbonylamino, benzoylamino, benzylcarbonylamino, and the like.

“Alkoxy” means a radical —OR where R is an alkyl as defined herein. Examples are methoxy, ethoxy, propoxy, butoxy and the like. “Alkoxycarbonyl” means a radical R—O—C(O)—, wherein R is an alkyl as defined herein. “Alkyl” means a linear saturated monovalent hydrocarbon radical of one to six carbon atoms or a branched saturated monovalent hydrocarbon radical of three to six carbon atoms.

Examples include methyl, ethyl, propyl, 2-propyl, n-butyl, iso-butyl, tert-butyl, pentyl, and the like. Preferred are C1-C3 alkyl groups, in particular ethyl and methyl. “Alkylsulfonyl” means a radical R—S(O)₂—, wherein R is alkyl as defined herein. “Alkylene” means a linear saturated divalent hydrocarbon radical of one to six carbon atoms or a branched saturated divalent hydrocarbon radical of three to six carbon atoms. Examples are methylene, ethylene, 2,2-dimethylethylene, propylene, 2-methylpropylene, butylen, pentylene, and the like.

“Aryl” means a monovalent monocyclic or bicyclic aromatic hydrocarbon radical which is optionally substituted independently with one or more substituents, preferably one, two or three substituents preferably selected from the group consisting of alkyl, hydroxy, alkoxy, haloalkyl, haloalkoxy, Y—C(O)—R (where Y is absent or an alkylene group and R is hydrogen, alkyl, haloalkyl, haloalkoxy, hydroxy, alkoxy, amino, monoalkylamino or dialkylamino), heteroalkyl, heteroalkyloxy, heteroalkylamino, halo, nitro, cyano, amino, monoalkylamino, dialkylamino, alkylsulfonylamino, heteroalkylsulfonylamino, sulfonamido, methylenedioxy, ethylenedioxy, heterocyclyl or heterocyclylalkyl. Monocyclic aryl groups, optionally substituted as described above, are preferred. More specifically, the term aryl includes, but is not limited to, phenyl optionally substituted independently with one, two or three substituents preferably selected from the group consisting of alkyl, hydroxy, alkoxy, haloalkyl, haloalkoxy, Y—C(O)—R (where Y is absent or an alkylene group and R is hydrogen, alkyl, haloalkyl, haloalkoxy, hydroxy, alkoxy, amino, monoalkylamino or dialkylamino), heteroalkyl, heteroalkyloxy, heteroalkylamino, halo, nitro, cyano, amino, monoalkylamino, dialkylamino, alkylsulfonylamino, heteroalkylsulfonylamino, sulfonamido, methylenedioxy, ethylenedioxy, heterocyclyl and heterocyclylalkyl. Particularly preferred aryl groups are substituted phenyl groups selected from the group consisting of chlorophenyl, methoxyphenyl, 2-fluorophenyl, 2,4-difluorophenyl, 1-naphthyl and 2-naphthyl. “Arylsulfonyl” means a radical R—S(O)₂—, wherein R is aryl as defined herein. “Aralkyl” refers to an aryl group as defined herein bonded directly through an alkylene group, e.g. benzyl.

“Aryloxy” means a radical -OR where R is an aryl as defined herein, e. g. phenoxy. “Aryloxycarbonyl” means a radical R—C(═O)— where R is aryloxy, e.g. phenoxycarbonyl. “Cycloalkyl”refers to a saturated monovalent cyclic hydrocarbon radical of three to seven ring carbons or more specifically those of the specific compounds listed in the enclosed tables or being described in the examples. It is understand that these radicals can be grouped also in a group covering only such radicals but of the first or the second priority application or of both priority applications e. g., cyclopropyl, cyclobutyl, cyclohexyl, 4-methyl-cyclohexyl, and the like.

“Cycloalkylalkyl” means a radical —R^{a}R^{b} where R^{a} is an alkylene group and R^{b} is cycloalkyl group as defined herein, e. g., cyclohexylmethyl, and the like.

“Substituted cycloalkyl” means a cycloalkyl radical as defined herein with one, two or three (preferably one) ring hydrogen atoms independently replaced by cyano or —Y—C(O)R (where Y is absent or an alkylene group and R is hydrogen, alkyl, haloalkyl, hydroxy, alkoxy, amino, monoalkylamino, dialkylamino, or optionally substituted phenyl) or more specifically those of the specific compounds listed in the enclosed tables or being described in the examples.

“Halo” means fluoro, chloro, bromo, or iodo, preferably fluoro and chloro.

“Haloalkyl” means alkyl substituted with one or more same or different halo atoms, e. g. —CH₂Cl, —CF₃, —CH₂CF₃, —CH₂CCl₃, and the like.

“Heteroalkyl” means an alkyl radical as defined herein wherein one, two or three hydrogen atoms have been replaced with a substituent independently selected from the group consisting of —OR^{a}, —N(O)ₙR^{b}R^{c} (where n is 0 or 1 if R^{b} and R^{c} are both independently alkyl, cycloalkyl or cycloalkylalkyl, and 0 if not) and —S(O)ₙR^{d} (where n is an integer from 0 to 2), with the understanding that the point of attachment of the heteroalkyl radical is through a carbon atom, wherein R^{a} is hydrogen, acyl, alkoxycarbonyl, alkyl, cycloalkyl, or cycloalkylalkyl; R^{b} and R^{c} are independently of each other hydrogen, acyl, alkoxycarbonyl, alkyl, cycloalkyl, cycloalkylalkyl, alkylsulfonyl, aminosulfonyl, mono- or dialkylaminosulfonyl, aminoalkyl, mono- or di-alkylaminoalkyl, hydroxyalkyl, alkoxyalkyl, hydroxyalkylsulfonyl or alkoxyalkylsulfonyl; and when n is 0, R^{d} is hydrogen, alkyl, cycloalkyl, cycloalkylalkyl or optionally substituted phenyl, and when n is 1 or 2, R^{d} is alkyl, cycloalkyl, cycloalkylalkyl, optionally substituted phenyl, amino, acylamino, monoalkylamino, or dialkylamino. Preferred heteroalkyl groups include hydroxyalkyl groups, preferably C1-C6 hydroxyalkyl groups. Representative examples include, but are not limited to, 2-hydroxyethyl, 2-hydroxy-propyl, 3-hydroxypropyl, 2-hydroxy-1-hydroxymethylethyl, 2-hydroxy-1-methylethyl, 2,3-dihydroxypropyl, 1-hydroxymethylethyl, 3-hydroxybutyl, 2,3-dihydroxybutyl, 2-hydroxy-1-methylpropyl, 3-hydroxy-1-(2-hydroxyethyl)-propyl, 2-aminoethyl, 3-aminopropyl, 2-methylsulfonylethyl, aminosulfonylmethyl, amino sulfonylethyl, aminosulfonylpropyl, methylaminosulfonylmethyl, methylaminosulfonylethyl, methylaminosulfonylpropyl, and the like. Particularly preferred heteroalkyl groups are 2-hydroxy-propyl, 3-hydroxy-1-(2-hydroxyethyl)-propyl or 2-hydroxy-1-methylethyl.

“Hydroxyalkyl” means an alkyl radical as defined herein, substituted with one or more, preferably one, two or three hydroxy groups, provided that the same carbon atom does not carry more than one hydroxy group. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 2-hydroxy-1-hydroxymethylethyl, 2,3-dihydroxybutyl, 3,4-dihydroxybutyl, 2-(hydroxymethyl)-3-hydroxypropyl, 3-hydroxy-1-(2-hydroxyethyl)-propyl and 2-hydroxy-1-methylethyl, preferably 2-hydroxyethyl, 2,3-dihydroxypropyl and 1-(hydroxymethyl)2-hydroxyethyl, more preferably 2-hydroxy-propyl, 3-hydroxy-1-(2-hydroxyethyl)-propyl and 2-hydroxy-1-methylethyl. Accordingly, as used herein, the term“hydroxyalkyl”is used to define a subset of heteroalkyl groups.

“Heteroalkylcarbonyl”means the group R^{a}—C (═O)—, where R^{a} is a heteroalkyl group. Representative examples include acetyloxymethylcarbonyl, aminomethylcarbonyl, 4-acetyloxy-2,2-dimethyl-butan-2-oyl, 2-amino-4-methyl-pentan-2-oyl, and the like.

“Heteroalkyloxy”means the group R^{a}—O—, where R^{a} is a heteroalkyl group. Representative examples include (Me-C(═O)—O—CH₂—O—, and the like.

“Heteroalkyloxycarbonyl” means the group R^{a}—C(═O), where R^{a} is heteroalkyloxy.

Representative examples include 1-acetyloxy-methoxycarbonyl (Me-C(═O)—OCH₂—O—C(═O)—) and the like.

“Heteroaryl”means a monovalent monocyclic or bicyclic radical of 5 to 12 ring atoms having at least one aromatic ring containing one, two, or three ring heteroatoms selected from the group consisting of N, O, or S, the remaining ring atoms being C, with the understanding that the attachment point of the heteroaryl radical will be on an aromatic ring. The heteroaryl ring is optionally substituted independently with one or more substituents, preferably one or two substituents, selected from the group consisting of alkyl, haloalkyl, heteroalkyl, hydroxy, alkoxy, halo, nitro or cyano. More specifically the term heteroaryl includes, but is not limited to, pyridyl, furanyl, thienyl, thiazolyl, isothiazolyl, triazolyl, imidazolyl, isoxazolyl, pyrrolyl, pyrazolyl, pyrimidinyl, benzofuranyl, tetrahydrobenzofuranyl, isobenzofuranyl, benzothiazolyl, benzoisothiazolyl, benzotriazolyl, indolyl, isoindolyl, benzoxazolyl, quinolyl, tetrahydroquinolinyl, isoquinolyl, benzimidazolyl, benzisoxazolyl or benzothienyl, imidazo[1,2-a]-pyridinyl, imidazo[2,1-b]thiazolyl, and derivatives thereof.

“Heteroaralkyl” means a radical —R^{a}R^{b} where R^{a} is an alkylene group and R^{b} is a heteroaryl group, e. g. pyridin-3-ylmethyl, imidazolylethyl, pyridinylethyl, 3-(benzofuran-2-yl)propyl, and the like.

“Heteroalkylsubstituted cycloalkyl” means a cycloalkyl radical as defined herein wherein one, two or three hydrogen atoms in the cycloalkyl radical have been replaced with a heteroalkyl group with the understanding that the heteroalkyl radical is attached to the cycloalkyl radical via a carbon-carbon bond. Representative examples include, but are not limited to, 1-hydroxymethylcyclopentyl, 2-hydroxymethylcyclohexyl, and the like.

“Heterosubstituted cycloalkyl” means a cycloalkyl radical as defined herein wherein one, two or three hydrogen atoms in the cycloalkyl radical have been replaced with a substituent independently selected from the group consisting of hydroxy, alkoxy, amino, acylamino, monoalkylamino, dialkylamino, oxo(C═O), imino, hydroximino (═NOH), NR′SO₂R^{d} (where R′ is hydrogen or alkyl and R^{d} is alkyl, cycloalkyl, hydroxyalkyl, amino, monoalkylamino or dialkylamino), —X—Y—C(O)R (where X is O or NR′, Y is alkylene or absent, R is hydrogen, alkyl, haloalkyl, alkoxy, amino, monoalkylamino, dialkylamino, or optionally substituted phenyl, and R′ is H or alkyl), or —S(O)ₙR (where n is an integer from 0 to 2) such that when n is 0, R is hydrogen, alkyl, cycloalkyl, cycloalkylalkyl optionally substituted phenyl or thienyl, and when n is 1 or 2, R is alkyl, cycloalkyl, cycloalkylalkyl, optionally substituted phenyl, thienyl, amino, acylamino, monoalkylamino or dialkylamino. Representative examples include, but are not limited to, 2-, 3-, or 4-hydroxycyclohexyl, 2-, 3-, or 4-aminocyclohexyl, 2-, 3-, or 4-methanesulfonamido-cyclohexyl, and the like, preferably 4-hydroxycyclohexyl, 2-aminocyclohexyl or 4-methanesulfonamido-cyclohexyl.

“Heterosubstituted cycloalkyl-alkyl” means a radical R^{a}R^{b}-where R^{a} is a heterosubstituted cycloalkyl radical and R^{b} is an alkylene radical.

“Heterocycloamino” means a saturated monovalent cyclic group of 4 to 8 ring atoms, wherein one ring atom is N and the remaining ring atoms are C. Representative examples include piperidine and pyrrolidine.

“Heterocyclyl” means a saturated or unsaturated non-aromatic cyclic radical of 3 to 8 ring atoms in which one or two ring atoms are heteroatoms selected from N, O, or S(O)ₙ (where n is an integer from 0 to 2), the remaining ring atoms being C, where one or two C atoms may optionally be replaced by a carbonyl group. The heterocyclyl ring may be optionally substituted independently with one, two, or three substituents selected from the group consisting of alkyl, haloalkyl, heteroalkyl, halo, nitro, cyano, cyanoalkyl, hydroxy, alkoxy, amino, monoalkylamino, dialkylamino, aralkyl, —(X)ₙ—C(O)R (where X is O or NR′, n is 0 or 1, R is hydrogen, alkyl, haloalkyl, hydroxy (when n is 0), alkoxy, amino, monoalkylamino, dialkylamino, or optionally substituted phenyl, and R′ is H or alkyl), -alkylene-C(O)R^{a} (where R^{a} is alkyl, OR or NR′R″ and R is hydrogen, alkyl or haloalkyl, and R′and R″are independently hydrogen or alkyl), or —S(O)ₙR (where n is an integer from 0 to 2) such that when n is 0, R is hydrogen, alkyl, cycloalkyl, or cycloalkylalkyl, and when n is 1 or 2, R is alkyl, cycloalkyl, cycloalkylalkyl, amino, acylamino, monoalkylamino, dialkylamino or heteroalkyl. More specifically the term heterocyclyl includes, but is not limited to, tetrahydropyranyl, piperidino, N-methylpiperidin-3-yl, piperazino, N-methylpyrrolidin-3-yl, 3-pyrrolidino, morpho lino, thiomorpholino, thiomorpholino-1-oxide, thiomorpholino-1,1-dioxide, 4-(1,1-dioxo-tetrahydro-2H-thiopyranyl), pyrrolinyl, imidazolinyl, N-methanesulfonyl-piperidin-4-yl, and the derivatives thereof.

“Heterocyclylalkyl” means a radical —R^{a}R^{b} where R^{a} is an alkylene group and R^{b} is a heterocyclyl group as defined above, e. g. tetrahydropyran-2-ylmethyl, 2- or 3-piperidinylmethyl, 3-(4-methyl-piperazin-1-yl)propyl and the like.

“(Heterocyclyl)(cycloalkyl)alkyl” means an alkyl radical wherein two hydrogen atoms have been replaced with a heterocyclyl group and a cycloalkyl group.

“(Heterocyclyl)(heteroaryl)alkyl” means an alkyl radical wherein two hydrogen atoms have been replaced with a heterocycyl group and a heteroaryl group.

“Amino” means a radical —NH₂.

“Monoalkylamino” means a radical —NHR where R is an alkyl, hydroxyalkyl, cycloalkyl, or cycloalkylalkyl group as defined above, e. g. methylamino, (1-methylethyl) amino, hydroxymethylamino, cyclohexylamino, cyclohexylmethylamino, cyclohexylethylamino, and the like.

“Dialkylamino” means a radical —NRR′ where R and R′ independently represent an alkyl, hydroxyalkyl, cycloalkyl, or cycloalkylalkyl group as defined herein. Representative examples include, but are not limited to dimethylamino, methylethylamino, di(1-methylethyl)amino, (methyl)(hydroxymethyl)amino, (cyclohexyl)(methyl)amino, (cyclohexyl)(ethyl)amino, (cyclohexyl)(propyl)amino, (cyclohexylmethyl)(methyl)amino, (cyclohexylmethyl)(ethyl)amino, and the like.

“Optionally substituted phenyl” means a phenyl ring which is optionally substituted independently with one or more substituents, preferably one, two or three substituents, more preferably two substituents selected from the group consisting of alkyl, hydroxy, alkoxy, haloalkyl, haloalkoxy, heteroalkyl, halo, nitro, cyano, amino, methylenedioxy, ethylenedioxy, and acyl, preferably halo, most preferably fluoro.

Thus, in a preferred embodiment, the p38 inhibitor for use in a pharmaceutical combination according to the invention is selected from the group consisting of pamapimod, acumapimod, losmapimod, dilmapimod, semapimod, AZD7624, ARRY-371797, LY2228820, R9111, PH-797804, BIRB 796, VX-702, VX-745, SB 239063, SB202190, SCIO 469, and BMS 582949 and a pharmaceutically acceptable salt thereof. More preferred is a p38 inhibitor for use in a pharmaceutical combination according to the invention selected from the group consisting of pamapimod, losmapimod, LY2228820, BMS 582949 or pharmaceutically acceptable salts and mixtures thereof or selected from the group consisting of pamapimod, losmapimod, LY2228820, BMS 582949 or pharmaceutically acceptable salts thereof, or selected from the group consisting of pamapimod, losmapimod, dilmapimod, R9111, LY2228820, BMS 582949 or pharmaceutically acceptable salts thereof, or selected from the group consisting of pamapimod, losmapimod, dilmapimod and R9111 or pharmaceutically acceptable salts thereof, more preferably pamapimod, losmapimod, or dilmapimod or pharmaceutically acceptable salts thereof, even more preferably pamapimod or dilmapimod or pharmaceutically acceptable salts thereof in particular pamapimod or a pharmaceutically acceptable salt thereof.

In a particularly preferred embodiment, the p38 inhibitor is pamapimod, having the chemical name 6-(2,4-Difluorophenoxy)-2-[3-hydroxy-1-(2-hydroxyethyl)-propylamino]-8-methyl-8H-pyrido[2,3-d]pyrimidin-7-one and the chemical formula III or a pharmaceutically acceptable salt thereof.

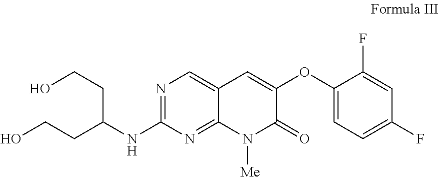 [SMILES: Cn1c(=O)c(Oc2ccc(F)cc2F)cc2cnc(NC(CCO)CCO)nc21]

Pamapimod and its synthesis are described e.g. in WO2008/151992 and in WO2002/064594 and in e.g. Hill R J, Dabbagh K, Phippard D, Li C, Suttmann R T, Welch M, Papp E, Song K W, Chang K C, Leaffer D, Kim Y-N, Roberts R T, Zabka T S, Aud D, Dal Porto J, Manning A M, Peng S L, Goldstein D M, and Wong B R; Pamapimod, a Novel p38 Mitogen-Activated Protein Kinase Inhibitor: Preclinical Analysis of Efficacy and Selectivity J Pharmacol Exp Ther. December 2008 327:610-619.

A further particularly preferred p38 inhibitor is losmapimod, having the chemical name 6-(5-((cyclopropylamino)carbonyl)-3-fluoro-2-methylphenyl)-N-(2,2-dimethylpropyl)-3-pyridinecarboxamide and the chemical formula IV or a pharmaceutically acceptable salt thereof.

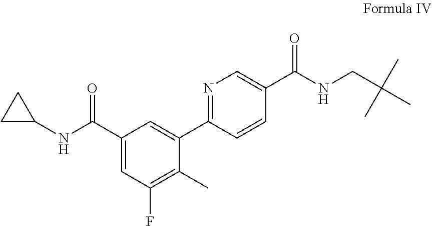 [SMILES: Cc1c(F)cc(C(=O)NC2CC2)cc1-c1ccc(C(=O)NCC(C)(C)C)cn1]

Losmapimod is described in e.g. Cheriyan J, Webb A J, Sarov-Blat L, Elkhawad M, Wallace S M, Maki-Petaja K M, Collier D J, Morgan J, Fang Z, Willette R N, Lepore J J, Cockcroft J R, Sprecher D L, Wilkinson I B. Inhibition of p38 mitogen-activated protein kinase improves nitric oxide-mediated vasodilatation and reduces inflammation in hypercholesterolemia. Circulation, 2011 Feb 8; 123(5):515-23.

Yet a further particularly preferred p38 inhibitor is LY2228820, having the chemical name 3-(2,2-Dimethylpropyl)-5-[4-(4-fluorophenyl)-2-(2-methyl-2-propanyl)-1H-imidazol-5 -yl]-3H-imidazo[4,5-b]pyridin-2-amine and the chemical formula V or a pharmaceutically acceptable salt thereof.

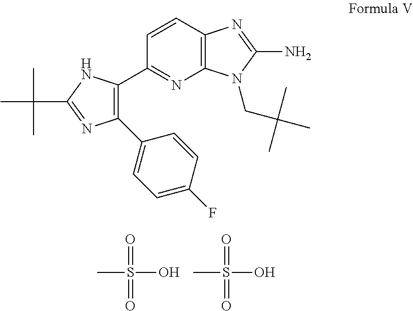

LY2228820 is described in e.g. Campbell R M, Anderson B D, Brooks N A, Brooks H B, Chan E M, De Dios A, Gilmour R, Graff J R, Jambrina E, Mader M, McCann D, Na S, Parsons S H, Pratt S E, Shih C, Stancato L F, Starling J J, Tate C, Velasco J A, Wang Y, Ye X S.

Characterization of LY2228820 dimesylate, a potent and selective inhibitor of p38 MAPK with antitumor activity. Mol Cancer Ther. 2014 Feb; 13(2):364-74.

Yet a further particularly preferred p38 inhibitor is BMS 582949, having the chemical name 4-(5-(cyclopropylcarbamoyl)-2-methylphenylamino)-5-methyl-N-propylpyrrolo[1,2-f][1,2,4]triazine-6-carboxamide and the chemical formula VI or a pharmaceutically acceptable salt thereof.

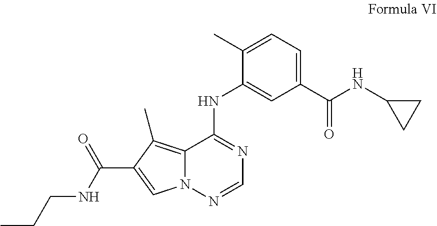 [SMILES: CCCNC(=O)c1cn2ncnc(Nc3cc(C(=O)NC4CC4)ccc3C)c2c1C]

BMS 582949 is described in e.g. Liu C, Lin J, Wrobleski S T, Lin S, Hynes J, Wu H, Dyckman A J, Li T, Wityak J, Gillooly K M, Pitt S, Shen D R, Zhang R F, McIntyre K W, Salter-Cid L, Shuster D J, Zhang H, Marathe P H, Doweyko A M, Sack J S, Kiefer S E, Kish K F, Newitt J A, McKinnon M, Dodd J H, Barrish J C, Schieven G L, Leftheris K. Discovery of 4-(5-(cyclopropylcarbamoyl)-2-methylphenylamino)-5-methyl-N-propylpyrrolo [1,2-f][1,2,4]triazine-6-carboxamide (BMS-582949), a clinical p38-alpha MAP kinase inhibitor for the treatment of inflammatory diseases. J Med Chem. 2010 Sep 23; 53(18):6629-39.

Acumapimod has the chemical name 3-[5-Amino-4-(3-cyanobenzoyl)-1H-pyrazol-1-yl]-N-cyclopropyl-4-methylbenzamide and is described in e.g De Buck S, Hueber W, Vitaliti A, Straube F, Emotte C, Bruin G, Woessner R. Population PK-PD Model for Tolerance Evaluation to the p38 MAP Kinase Inhibitor BCT197. CPT Pharmacometrics Syst Pharmacol. 2015 Dec; 4(12):691-700 , and is represented by the structural formula indicated below:

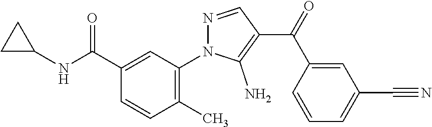 [SMILES: Cc1ccc(C(=O)NC2CC2)cc1-n1ncc(C(=O)c2cccc(C#N)c2)c1N]

Dilmapimod is described in e.g. Christie J D, Vaslef S, Chang P K, May A K, Gunn S R, Yang S, Hardes K, Kahl L, Powley W M, Lipson D A, Bayliffe A I, Lazaar A L. A Randomized Dose-Escalation Study of the Safety and Anti-Inflammatory Activity of the p38 Mitogen-Activated Protein Kinase Inhibitor Dilmapimod in Severe Trauma Subjects at Risk for Acute Respiratory Distress Syndrome. Crit Care Med. 2015 Sep; 43(9):1859-69, and is represented by the structural formula indicated below:

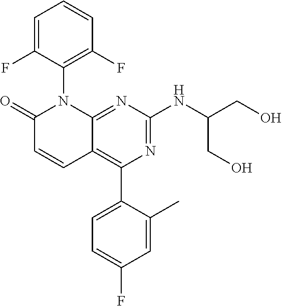 [SMILES: Cc1cc(F)ccc1-c1nc(NC(CO)CO)nc2c1ccc(=O)n2-c1c(F)cccc1F]

Semapimod is described in e.g. Bianchi, M.; Ulrich, P.; Bloom, O.; Meistrell m, M. , I. I.; Zimmerman, G. A.; Schmidtmayerova, H.; Bukrinsky, M.; Donnelley, T.; Bucala, R.; Sherry, B.; Manogue, K. R.; Tortolani, A. J.; Cerami, A.; Tracey, K. J. (Mar 1995). Molecular Medicine (Cambridge, Mass.). 1 (3): 254-266 or in e.g. Wang J, Grishin A V, Ford H R. Experimental Anti-Inflammatory Drug Semapimod Inhibits TLR Signaling by Targeting the TLR Chaperone gp96. J Immunol. 2016 Jun 15; 196(12):5130-7 and is represented by the structural formula as indicated below:

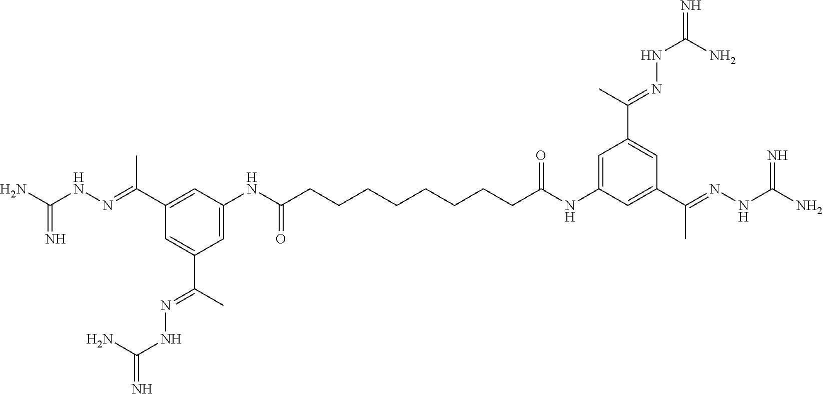 [SMILES: C/C(=N\NC(=N)N)c1cc(NC(=O)CCCCCCCCC(=O)Nc2cc(/C(C)=N/NC(=N)N)cc(/C(C)=N/NC(=N)N)c2)cc(/C(C)=N/NC(=N)N)c1]

AZD7624 is described in e.g. Patel N, Cunoosamy D, Hegelund-Myrback T, Pehrson R, Taib Z, Jansson P, Lundin S, Greenaway S, Clarke G, Siew L. AZD7624, an inhaled p38 inhibitor for COPD, attenuates lung and systemic inflammation after LPS Challenge in humans. Eur

Resp J. DOI: 10.1183/13993003.1 Sept 2015, and is represented by the structural formula as indicated below:

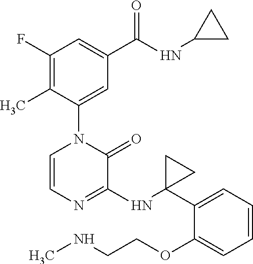 [SMILES: CNCCOc1ccccc1C1(Nc2nccn(-c3cc(C(=O)NC4CC4)cc(F)c3C)c2=O)CC1]

ARRY-371797 is described in e.g. Muchir A, Wu W, Choi J C, Iwata S, Morrow J, Homma S, Worman H J. Abnormal p38-alpha mitogen-activated protein kinase signaling in dilated cardiomyopathy caused by lamin A/C gene mutation. Hum Mol Genet. 2012 Oct 1; 21(19):4325-33, and is represented by the structural formula as indicated below:

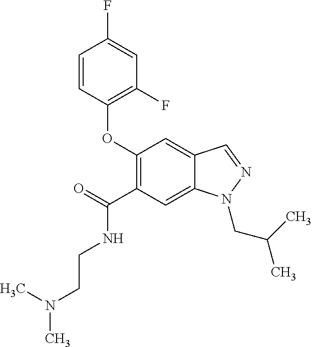 [SMILES: CC(C)Cn1ncc2cc(Oc3ccc(F)cc3F)c(C(=O)NCCN(C)C)cc21]

R9111 and its synthesis is described in WO2005/047284 and in e.g. Hill R J, Dabbagh K, Phippard D, Li C, Suttmann R T, Welch M, Papp E, Song K W, Chang K C, Leaffer D, Kim Y-N, Roberts R T, Zabka T S, Aud D, Dal Porto J, Manning A M, Peng S L, Goldstein D M, and Wong B R; Pamapimod, a Novel p38 Mitogen-Activated Protein Kinase Inhibitor: Preclinical Analysis of Efficacy and Selectivity J Pharmacol Exp Ther. December 2008 327:610-619 and is represented by the structural formula as indicated below:

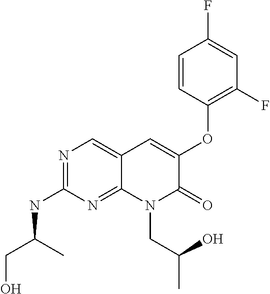 [SMILES: C[C@H](O)Cn1c(=O)c(Oc2ccc(F)cc2F)cc2cnc(N[C@@H](C)CO)nc21]

PH-797804 is described in e.g. Xing L, Devadas B, Devraj R V, Selness S R, Shieh H, Walker J K, Mao M, Messing D, Samas B, Yang J Z, Anderson G D, Webb E G, Monahan J B. Discovery and characterization of atropisomer PH-797804, a p38 MAP kinase inhibitor, as a clinical drug candidate. ChemMedChem. 2012 Feb 6; 7(2):273-80, and is represented by the structural formula indicated below:

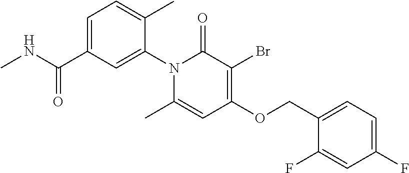 [SMILES: CNC(=O)c1ccc(C)c(-n2c(C)cc(OCc3ccc(F)cc3F)c(Br)c2=O)c1]

BIRB 796 is described in e.g. Dietrich J, Hulme C, Hurley L H. The design, synthesis, and evaluation of 8 hybrid DFG-out allosteric kinase inhibitors: a structural analysis of the binding interactions of Gleevec, Nexavar, and BIRB-796. Bioorg Med Chem. 2010 Aug 1; 18(15):5738-48, and is represented by the structural formula indicated below:

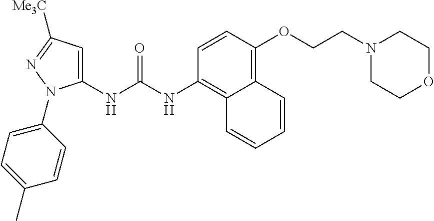

VX-702 is described in e.g. Damjanov N, Kauffman R S, Spencer-Green G T. Efficacy, pharmacodynamics, and safety of VX-702, a novel p38 MAPK inhibitor, in rheumatoid arthritis: results of two randomized, double-blind, placebo-controlled clinical studies. Arthritis Rheum. 2009 May; 60(5):1232-41, and is represented by the structural formula indicated below:

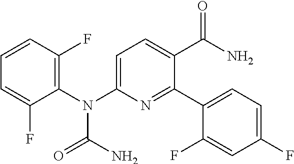 [SMILES: NC(=O)c1ccc(N(C(N)=O)c2c(F)cccc2F)nc1-c1ccc(F)cc1F]

VX-745 is described in e.g. Duffy J P, Harrington E M, Salituro F G, Cochran J E, Green J, Gao H, Bemis G W, Evindar G, Galullo V P, Ford P J, Germann U A, Wilson K P, Bellon S F, Chen G, Taslimi P, Jones P, Huang C, Pazhanisamy S, Wang Y M, Murcko M A, Su M S. The Discovery of VX-745: A Novel and Selective p38-alpha Kinase Inhibitor. ACS Med Chem Lett. 2011 Jul 28; 2(10):758-63, and is represented by the structural formula indicated below:

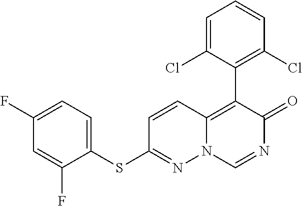 [SMILES: O=c1ncn2nc(Sc3ccc(F)cc3F)ccc2c1-c1c(Cl)cccc1Cl]

SB239063 is described in e.g. Strassburger M, Braun H, Reymann K G. Anti-inflammatory treatment with the p38 mitogen-activated protein kinase inhibitor SB239063 is neuroprotective, decreases the number of activated microglia and facilitates neurogenesis in oxygen-glucose-deprived hippocampal slice cultures. Eur J Pharmacol. 2008 Sep 11; 592(1-3):55-61, and is represented by the structural formula indicated below:

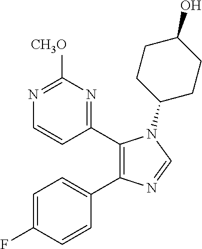

SB202190 is described in e.g. Hirosawa M, Nakahara M, Otosaka R, Imoto A, Okazaki T, Takahashi S. The p38 pathway inhibitor SB202190 activates MEK/MAPK to stimulate the growth of leukemia cells. Leuk Res. 2009 May; 33(5):693-9, and is represented by the structural formula indicated below:

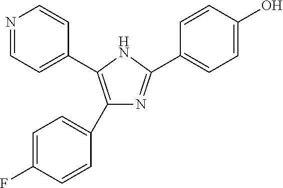 [SMILES: Oc1ccc(-c2nc(-c3ccc(F)cc3)c(-c3ccncc3)[nH]2)cc1]

SCI0469 is described in e.g. Sokol L, Cripe L, Kantarjian H, Sekeres M A, Parmar S, Greenberg P, Goldberg S L, Bhushan V, Shammo J, Hohl R, Verma A, Garcia-Manero G, Li Y P, Lowe A, Zhu J, List AF. Randomized, dose-escalation study of the p38-alpha MAPK inhibitor SCID-469 in patients with myelodysplastic syndrome. Leukemia. 2013 Apr; 27(4):977-80, and is represented by the structural formula indicated below:

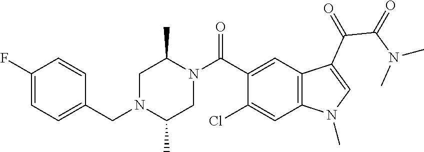 [SMILES: C[C@@H]1CN(Cc2ccc(F)cc2)[C@@H](C)CN1C(=O)c1cc2c(C(=O)C(=O)N(C)C)cn(C)c2cc1Cl]

Pharmaceutical Combinations

As outlined above, in a first aspect, the present invention provides a pharmaceutical combination comprising:

(a) a PPAR agonist;

(b) a p38 kinase inhibitor; and optionally

(c) one or more pharmaceutically acceptable diluents, excipients or carriers for use in a method of preventing or treating fibrotic diseases or disorders in a subject.

Useful PPAR agonists are as defined above. In one embodiment, said PPAR agonist is activating PPAR gamma and/or PPAR alpha. In a preferred embodiment, said PPAR agonist is selected from the group consisting of pioglitazone, rosiglitazone, troglitazone, fenofibrate, bezafibrate and pharmaceutically acceptable salts thereof. In a more preferred embodiment, said PPAR agonist is a PPAR gamma agonist, preferably a PPAR gamma agonist selected from the group consisting of pioglitazone, rosiglitazone and troglitazone or pharmaceutically acceptable salts thereof, in particular pioglitazone or a pharmaceutically acceptable salt thereof. In a particularly preferred embodiment, said PPAR agonist is pioglitazone hydrochloride.

Useful p38 kinase inhibitors are as defined above. In a preferred embodiment, said p38 kinase inhibitors are inhibiting p38-alpha, p38-beta, p38-gamma or p38-delta or combinations thereof, preferably inhibiting p38-alpha and/or p38-beta, more preferably inhibiting p38-alpha. Further useful p38 kinase inhibitors are compunds of the formula I or II, or pharmaceutically acceptable salts thereof, as defined supra. Further useful p38 kinase inhibitors are p38 kinase inhibitors selected from the group consisting of pamapimod, acumapimod, losmapimod, dilmapimod, semapimod, AZD7624, ARRY-371797, LY2228820, R9111, PH-797804, BIRB 796, VX-702, VX-745, SB 239063, SB202190, SCIO 469, BMS 582949, and pharmaceutically acceptable salts thereof, in particular pamapimod, losmapimod, LY2228820, BMS 582949 or pharmaceutically acceptable salts and mixtures thereof, or selected from the group consisting of pamapimod, acumapimod, losmapimod, dilmapimod, semapimod, AZD7624, ARRY-371797, LY2228820, R9111, PH-797804, BIRB 796, VX-702, VX-745, SB 239063, SB202190, SCIO 469, BMS 582949, and pharmaceutically acceptable salts thereof, in particular pamapimod, losmapimod, LY2228820, BMS 582949 or pharmaceutically acceptable salts thereof, preferably pamapimod, losmapimod, dilmapimod or R9111 or pharmaceutically acceptable salts thereof, more preferably pamapimod or dilmapimod or pharmaceutically acceptable salts thereof, more particularly pamapimod or a pharmaceutically acceptable salt thereof.

A pharmaceutical combination according to the invention is for example a combined preparation or a pharmaceutical composition, for simultaneous, separate or sequential use. The term “combined preparation” as used herein defines especially a “kit of parts” in the sense that said PPAR agonist and said p38 inhibitor can be dosed independently, either in separate form e.g. as separate tablets or by use of different fixed combinations with distinguished amounts of the active ingredients. The ratio of the amount of PPAR agonist to the amount of p38 inhibitor to be administered in the combined preparation can be varied, e.g. in order to cope with the needs of a patient sub-population to be treated or the needs of a single patient, which needs can be different due to age, sex, body weight, etc. of a patient. The individual parts of the combined preparation (kit of parts) can be administered simultaneously or sequentially, i.e. chronologically staggered, e.g. at different time points and with equal or different time intervals for any part of the kit of parts.

The term “pharmaceutical composition” refers to a fixed-dose combination (FDC) that includes the PPAR agonist and the p38 inhibitor combined in a single dosage form, having a predetermined combination of respective dosages.

The pharmaceutical combination further may be used as add-on therapy. As used herein, “add-on” or “add-on therapy” means an assemblage of reagents for use in therapy, the subject receiving the therapy begins a first treatment regimen of one or more reagents prior to beginning a second treatment regimen of one or more different reagents in addition to the first treatment regimen, so that not all of the reagents used in the therapy are started at the same time. For example, adding p38 inhibitor therapy to a patient already receiving PPAR agonist therapy and vice versa.

In a preferred embodiment, the pharmaceutical combination according to the invention is a combined preparation.

In a further preferred embodiment, the pharmaceutical combination according to the invention is a pharmaceutical composition, i.e. a fixed-dose combination.

The amount of the PPAR agonist and the p38 inhibitor to be administered will vary depending upon factors such as the particular compound, disease condition and its severity, according to the particular circumstances surrounding the case, including, e.g., the specific PPAR agonist being administered, the route of administration, the condition being treated, the target area being treated, and the subject or host being treated.

In one embodiment, the invention provides a pharmaceutical combination comprising a PPAR agonist and a p38 inhibitor, wherein said PPAR agonist and said p38 inhibitor are present in a therapeutically effective amount.

In a preferred embodiment, the invention provides a pharmaceutical combination comprising a PPAR agonist and a p38 inhibitor, wherein said PPAR agonist and said p38 inhibitor produce an additive therapeutic effect i.e. wherein said PPAR agonist and said p38 inhibitor are present in an amount producing an additive therapeutic effect.

As used herein, the term “additive” means that the effect achieved with the pharmaceutical combinations of this invention is approximately the sum of the effects that result from using the agents, namely the PPAR agonist and the p38 inhibitor, as a monotherapy. Advantageously, an additive effect provides for greater efficacy at the same doses, and may lead to longer duration of response to the therapy.

In a further preferred embodiment, the invention provides a pharmaceutical combination comprising a PPAR agonist and a p38 inhibitor, wherein said PPAR agonist and said p38 inhibitor produce a synergistic therapeutic effect, i.e. wherein said PPAR agonist and said p38 inhibitor are present in an amount producing a synergistic therapeutic effect.

In a further more preferred embodiment, the invention provides a pharmaceutical combination comprising a PPAR agonist and a p38 inhibitor, wherein said PPAR agonist and said p38 inhibitor produce a synergistic therapeutic effect in relation to the regulation of the expression of interleukin/interleukin receptor and/or TNF/TNF receptor genes, and/or C-C and C-X-C motif chemokine gene families, more particular wherein said PPAR agonist and said p38 inhibitor produce a synergistic therapeutic effect in relation to the regulation of the expression of one or more interleukin/interleukin receptor genes selected from the group consisting of interleukin 6, interleukin 11, interleukin 12B, and interleukin 18 receptor 1 and/or in relation to the regulation of the expression of TNF/TNF receptor genes selected from the group consisting of TNF receptor superfamily member 11b, TNF superfamily member 9, TNF receptor superfamily member 10b, and TNF superfamily member 18 and/or in relation to the regulation of the expression of the C-C and C-X-C motif chemokine genes selected from the group consisting of C-C motif chemokine ligand 7, C-C motif chemokine ligand 4, C-C motif chemokine ligand 3, C-C motif chemokine ligand 12, C-C motif chemokine ligand 2, C-C motif chemokine ligand 8, C-C motif chemokine ligand 24, C-X-C motif chemokine ligand 10, C-X-C motif chemokine ligand 5, C-X-C motif chemokine ligand 3, even more particular wherein said PPAR agonist and said p38 inhibitor produce a synergistic therapeutic effect in relation to the regulation of the expression of one or more interleukin/interleukin receptor genes selected from the group consisting of interleukin 6, interleukin 11, interleukin 12B, and interleukin 18 receptor 1 and/or in relation to the regulation of the expression of TNF/TNF receptor genes selected from the group consisting of TNF receptor superfamily member 1 lb, TNF superfamily member 9, TNF receptor superfamily member 10b, and TNF superfamily member 18.

As used herein, the term “synergistic” means that the effect achieved with the pharmaceutical combinations of this invention is greater than the sum of the effects that result from using the agents, namely the PPAR agonist and the p38 inhibitor, as a monotherapy. Advantageously, such synergy provides greater efficacy at the same doses and may lead to longer duration of response to the therapy.

In one embodiment, the invention provides a pharmaceutical combination comprising a p38 inhibitor and a PPAR agonist, wherein the amount of said PPAR agonist in the combination is from about 0.1 to about 45 mg, from about 0.1 to about 30 mg or from about 0.1 to about 15 mg. Where said PPAR agonist is in the form of a pharmaceutically acceptable salt, the amounts of PPAR agonist provided herein are calculated on the basis of the respective free base.

In a preferred embodiment, the invention provides a pharmaceutical combination comprising a p38 inhibitor and pioglitazone or a pharmaceutically acceptable salt thereof, wherein the amount of pioglitazone or a pharmaceutically acceptable salt thereof in the combination is below the dose typically needed for the treatment of diabetes with pioglitazone or a pharmaceutically acceptable salt thereof.

In one embodiment, the invention provides a pharmaceutical combination comprising a p38 inhibitor and a PPAR agonist, wherein the amount of said p38 inhibitor in the combination is from about 1 to about 500 mg or from about 1 to about 450 mg or from about 1 to about 400 mg or from about 1 to about 350 mg or from about 1 to about 300 mg or from about 1 to about 250 mg or from about 1 to about 200 mg or from about 1 to about 150 mg or from about 1 to about 125 mg or from about 1 to about 100 mg or from about 10 to about 125 mg or from about 10 to about 100 mg or from about 20 to about 100 mg or from about 30 to about 100 mg or from about 40 to about 100 mg or from about 50 to about 100 mg.

In a preferred embodiment, the pharmaceutical combination of the invention is a pharmaceutical composition (i.e. a fixed-dose combination, as outlined above). In one embodiment, the pharmaceutical combination of the invention is a pharmaceutical composition and includes other medicinal or pharmaceutical agents, e.g., one or more pharmaceutically acceptable diluents, excipients or carriers.

Modes of Administration and Treatment

The terms “treatment”/“treating” as used herein includes: (1) delaying the appearance of clinical symptoms of the state, disorder or condition developing in an animal, particularly a mammal and especially a human, that may be afflicted with or predisposed to the state, disorder or condition but does not yet experience or display clinical or subclinical symptoms of the state, disorder or condition; (2) inhibiting the state, disorder or condition (e.g. arresting, reducing or delaying the progression of the disease, or a relapse thereof in case of maintenance treatment, of at least one clinical or subclinical symptom thereof); and/or (3) relieving the condition (i.e. causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

Preventive treatments comprise prophylactic treatments. In preventive applications, the pharmaceutical combination of the invention is administered to a subject suspected of having, or being at risk for developing fibrotic diseases or disorders. In therapeutic applications, the pharmaceutical combination of the invention is administered to a subject such as a patient already suffering from fibrotic diseases or disorders, in an amount sufficient to cure or at least partially arrest the symptoms of the disease. Amounts effective for this use will depend on the severity and course of the disease, previous therapy, the subject's health status and response to the drugs, and the judgment of the treating physician. In the case wherein the subject's condition does not improve, the pharmaceutical combination of the invention may be administered chronically, which is, for an extended period of time, including throughout the duration of the subject's life in order to ameliorate or otherwise control or limit the symptoms of the subject's disease or condition.

In the case wherein the subject's status does improve, the pharmaceutical combination of the invention may be administered continuously; alternatively, the dose of drugs being administered may be temporarily reduced or temporarily suspended for a certain length of time (i.e., a “drug holiday”).

The pharmaceutical combination according to the invention is, preferably, suitable for oral, topical, injectable, ocular, local ocular (e.g., subconjunctival, intravitreal, retrobulbar or intracameral), systemic (i.e. enteral or parenteral) administration or suitable for administration by inhalation i.e. the combination is administered locally to the lung. More preferably the pharmaceutical combination according to the invention is suitable for oral, topical, injectable administration and/or administration by inhalation, most preferably suitable for oral administration to a subject and comprises a therapeutically effective amount of the active ingredient(s) and optionally one or more suitable pharmaceutically acceptable diluents, excipients or carriers.

If not indicated otherwise, a pharmaceutical combination according to the invention is prepared in a manner known per se, e.g. by means of conventional mixing, granulating, coating, dissolving or lyophilizing processes. In preparing a combination for an oral dosage form, any of the usual pharmaceutical media may be employed, carriers, diluents, granulating agents, lubricants, binders, disintegrating agents and the like. Because of their ease of administration, tablets and capsules represent the most advantageous oral dosage unit forms, in which case solid pharmaceutical carriers are obviously employed.

In a preferred embodiment, the pharmaceutical combination according to the invention is a combination for oral administration. As indicated above, said pharmaceutical combination for oral administration is preferably a pharmaceutical composition, i.e. a fixed-dose combination.

In one embodiment, the pharmaceutical combination according to the invention is a combination for topical administration. As indicated above, said pharmaceutical combination for topical administration is preferably a pharmaceutical composition, i.e. a fixed-dose combination.

In another preferred embodiment, the pharmaceutical combination according to the invention is a combination for injectable administration. As indicated above, said pharmaceutical combination for injectable administration is preferably a pharmaceutical composition, i.e. a fixed-dose combination.

In one embodiment, the pharmaceutical combination according to the invention is a combination for local ocular administration. As indicated above, said pharmaceutical combination for local ocular administration is preferably a pharmaceutical composition, i.e. a fixed-dose combination.

In another preferred embodiment, the pharmaceutical combination according to the invention is a combination for systemic, i.e. enteral or parenteral administration. As indicated above, said pharmaceutical combination for systemic administration is preferably a pharmaceutical composition, i.e. a fixed-dose combination.

In another preferred embodiment, the pharmaceutical combination according to the invention is a combination for administration by inhalation, for example as a nasal spray, or dry powder or aerosol inhalers. For delivery by inhalation, the active compounds are preferably in the form of microparticles. They may be prepared by a variety of techniques, including spray-drying, freeze-drying and micronisation. Aerosol generation can be carried out using, for example, pressure-driven jet atomizers or ultrasonic atomizers, preferably using propellant-driven metered aerosols or propellant-free administration of micronized active compounds from, for example, inhalation capsules or other “dry powder” delivery systems. Microparticles for delivery by inhalation may be formulated with excipients that aid delivery and release. For example, in a dry powder formulation, microparticles may be formulated with large carrier particles that aid flow from the DPI into the lung. Suitable carrier particles are known, and include lactose particles; they may have a mass median aerodynamic diameter of greater than 90 μm.

The active compounds may be dosed as described depending on the inhaler system used. In addition to the active compounds, the administration forms may additionally contain excipients, such as, for example, propellants (e.g. Frigen in the case of metered aerosols), surface-active substances, emulsifiers, stabilizers, preservatives, flavorings, fillers (e.g. lactose in the case of powder inhalers) or, if appropriate, further active compounds.

For the purposes of inhalation, a large number of systems are available with which aerosols of optimum particle size can be generated and administered, using an inhalation technique which is appropriate for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhalers for example as described EP-A-0505321). Additionally, the combination of the invention may be delivered in multi-chamber devices thus allowing for separate storage and dosing of the PPAR agonist and the p38 inhibitor according to the invention.

In a more preferred embodiment the pharmaceutical combination according to the invention is administered orally, topically, by injection or by inhalation, even more preferably orally to the subject.

In a preferred embodiment the fibrotic diseases or disorders is lung fibrosis and the pharmaceutical combination according to the invention is a combination for oral administration, i.e. is administered orally. As indicated above, said pharmaceutical combination for oral administration is preferably a pharmaceutical composition, i.e. a fixed-dose combination.

In a further preferred embodiment the fibrotic diseases or disorders is lung fibrosis and the pharmaceutical combination according to the invention is a combination for administration by inhalation, i.e. is administered to the lung. As indicated above, said pharmaceutical combination for administration by inhalation is preferably a pharmaceutical composition, i.e. a fixed-dose combination.

In one embodiment the fibrotic diseases or disorders is liver fibrosis and the pharmaceutical combination according to the invention is a combination for oral administration, i.e. is administered orally. As indicated above, said pharmaceutical combination for oral administration is preferably a pharmaceutical composition, i.e. a fixed-dose combination.

In one embodiment the fibrotic diseases or disorders is kidney fibrosis and the pharmaceutical combination according to the invention is a combination for oral administration, i.e. is administered orally. As indicated above, said pharmaceutical combination for oral administration is preferably a pharmaceutical composition, i.e. a fixed-dose combination.

In one embodiment the fibrotic diseases or disorders is ocular fibrosis and the pharmaceutical combination according to the invention is a combination for oral, injection or topical administration, i.e. is administered orally, by injection or topically. As indicated above, said pharmaceutical combination for oral, injection or topical administration is preferably a pharmaceutical composition, i.e. a fixed-dose combination.

In one embodiment the fibrotic diseases or disorders is cutaneous fibrosis and the pharmaceutical combination according to the invention is a combination for oral or topical administration, i.e. is administered orally or topically. As indicated above, said pharmaceutical combination for oral or topical administration is preferably a pharmaceutical composition, i.e. a fixed-dose combination.

A pharmaceutical combination for oral or systemic i.e. enteral or parenteral administration is, for example, a unit dosage form, such as a tablet, a capsule or a suppository.

In one embodiment, the invention provides a pharmaceutical composition comprising a PPAR agonist, such as pioglitazone and a p38 inhhibitor, such as pamapimod and at least one pharmaceutically acceptable carrier, wherein the composition is a solution or a suspension for ocular administration (i.e. eye drops), or an ophthalmic ointment.

In one embodiment, the invention provides a pharmaceutical composition comprising a PPAR agonist, such as pioglitazone and a p38 inhhibitor, such as pamapimod and at least one pharmaceutically acceptable carrier, wherein the composition is a tablet or a capsule, preferably a tablet.

The unit content of active ingredients in an individual dose need not in itself constitute a therapeutically effective amount, since such an amount can be reached by the administration of a plurality of dosage units. A composition according to the invention may contain, e.g., from about 10% to about 100% of the therapeutically effective amount of the active ingredients.

Where the pharmaceutical combination according to the invention is a combined preparation, said PPAR agonist need not be administered in the same form as said p38 inhibitor. As an example, the PPAR agonist may be administered as a powder by inhalation, while the p38 inhibitor may be administered orally as a tablet or vice versa.

In some embodiments the pharmaceutical combination of the invention is administered to the subject in a dose that comprises a dose of a PPAR agonist which is below the dose needed for the treatment of diabetes using said PPAR agonist. In some embodiments the pharmaceutical combination of the invention is administered to the subject in a dose that comprises a dose of a PPAR agonist which is a factor of 3-9 fold lower than the top dose evaluated and tested for the treatment of diabetes, in particular a factor of 3-9 fold lower than the top dose evaluated and tested for the treatment of diabetes in human. The top dose evaluated and tested for the treatment of diabetes in human, e.g for a PPAR gamma agonists such as pioglitazone hydrochloride, is usually in the range of about 15-45 mg/day. In some embodiments at the PPAR agonist dose used, the side effects seen in the treatment of diabetes using said PPAR agonist are reduced or not present.

In some embodiments the pharmaceutical combination of the invention is administered to the subject in a dose that comprises a dose of a PPAR agonist which is below the active dose for therapeutically relevant antidiabetic or anti-dyslipidemic effect of the PPAR agonist, in particular a dose that is below the active dose for antidiabetic or anti-dyslipidemic effect of the PPAR agonist in human.

A typical dosing regimen of pioglitazone or a pharmaceutically acceptable salt thereof in the treatment of diabetes includes 15 to 45 mg pioglitazone once-daily.

In some embodiments, the pharmaceutical combination of the invention is administered orally to a human in a dose comprising a dose of a PPAR agonist, usually PPAR gamma agonists, PPAR alpha agonists and/or PPAR alpha/gamma dual agonists, preferably a PPAR gamma agonist and/or a PPAR alpha agonist, more preferably a PPAR gamma agonist and/or a PPAR alpha agonist selected from the group consisting of pioglitazone, rosiglitazone, troglitazone, feonofibrate, bezafibrate and pharmaceutically acceptable salts thereof, even more preferably a PPAR gamma agonist, yet more preferably pioglitazone or a pharmaceutically acceptable salt thereof, most preferably pioglitazone hydrochloride of 0.1-45 mg/day, preferably 0.1-10 mg/day, more preferably about 5 mg/day; and comprising a dose of a p38 inhibitor, e.g. a compound of formula I or II, in particular a compound of formula I, preferably a p38 inhibitor selected from the group consisting of pamapimod, acumapimod, losmapimod, dilmapimod, semapimod, AZD7624, ARRY-371797, LY2228820, R9111, PH-797804, BIRB 796, VX-702, VX-745 SB 239063, SB202190, SCIO 469, and BMS 582949 or a pharmaceutically acceptable salt thereof, more preferably pamapimod or a pharmaceutically acceptable salt thereof of 1-500 mg/day, preferably 10-250 mg/day, more preferably 25-150 mg/day, most preferably about 75 mg/day.

Dosing Regimen

An exemplary treatment regime entails administration once daily, twice daily, or thrice daily every second day, preferably once daily and/or twice daily . The combination of the invention is usually administered on multiple occasions. Intervals between single dosages can be, for example, less than a day, daily, or every second day. The combination of the invention may be given as a continous uninterrupted treatment. The combination of the invention may also be given in a regime in which the subject receives cycles of treatment interrupted by a drug holiday or period of non-treatment. Thus, the combination of the invention may be administered according to the selected intervals above for a continuous period of one week or a part thereof, for two weeks, for three weeks, for four weeks, for five weeks or for six weeks and then stopped for a period of one week, or a part thereof, for two weeks, for three weeks, for four weeks, for five weeks, or for six weeks. The combination of the treament interval and the non-treatment interval is called a cycle. The cycle may be repeated one or more times. Two or more different cycles may be used in combination for repeating the treatment one or more times. Intervals can also be irregular and guided either by worseining or improvement in the condition of the patient indicated by appearance or remission of symptoms or objective evidence of disease appearance or remission. In such case, therapy may be started and suspended as needed, and only restarted when symptoms or objective measures indicate the return of disease. In a preferred embodiment, the pharmaceutical combination according to the invention is administered once daily.

Kits/Articles of Manufacture

In one aspect, the present invention also provides a kit for use in a method of preventing or treating fibrotic diseases or disorders in a subject, comprising a pharmaceutical combination disclosed herein, and instructions for using the kit. Preferred PPAR agonists and preferred p38 kinase inhibitors comprised by said pharmaceutical combination are as described above.

In some embodiments, kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. In other embodiments, the containers are formed from a variety of materials such as glass or plastic. The articles of manufacture provided herein generally will comprise one or more pharmaceutical combination disclosed herein and packaging materials. Examples of pharmaceutical packaging materials include, but are not limited to, blister packs, bottles, tubes, inhalers, pumps, bags, vials, containers, syringes, and any packaging material suitable for a selected composition and intended mode of administration and treatment.

Preventing or Treating Fibrotic Diseases or Disorders

In one aspect, the present invention provides a pharmaceutical combination described herein, i.e. a pharmaceutical combination comprising:

(a) a PPAR agonist;

(b) a p38 kinase inhibitor; and optionally

(c) one or more pharmaceutically acceptable diluents, excipients or carriers for use in a method of preventing or treating fibrotic diseases or disorders in a subject.

Also provided is the use of a pharmaceutical combination described herein for the manufacture of a medicament for preventing or treating fibrotic diseases or disorders in a subject.

Also provided is the use of a pharmaceutical combination described herein for preventing or treating fibrotic diseases or disorders in a subject.

Also provided is a method of preventing or treating fibrotic diseases or disorders in a subject, comprising administering to said subject a therapeutically effective amount of a pharmaceutical combination as described herein.

In a preferred embodiment, the present invention provides a pharmaceutical combination described herein for use in a method of preventing or treating fibrotic diseases or disorders in a subject, wherein said fibrotic disease or disorder is selected from the group consisting of lung fibrosis, liver fibrosis, kidney fibrosis, cardiac fibrosis, ocular fibrosis or cutaneous fibrosis.

In a more preferred embodiment, the present invention provides a pharmaceutical combination described herein for use in a method of preventing or treating lung fibrosis.

In an even more preferred embodiment, the present invention provides a pharmaceutical combination described herein for use in a method of preventing or treating lung fibrosis, wherein the lung fibrosis is selected from the group consisting of idiopathic pulmonary fibrosis (IPF), familial interstitial pulmonary fibrosis, nonspecific interstitial pneumonia (NSIP), cryptogenic organizing pneumonia (COP), sarcoidosis, and asbestosis.

In a further even more preferred embodiment, the present invention provides a pharmaceutical combination described herein for use in a method of preventing or treating lung fibrosis, wherein the lung fibrosis is selected from the group consisting of idiopathic pulmonary fibrosis (IPF), familial interstitial pulmonary fibrosis, nonspecific interstitial pneumonia (NSIP), cryptogenic organizing pneumonia (COP), sarcoidosis, chronic obstructive pulmonary disease (COPD), and asbestosis.

In a particular preferred embodiment, the present invention provides a pharmaceutical combination described herein for use in a method of preventing or treating lung fibrosis, wherein the lung fibrosis is selected from the group consisting of idiopathic pulmonary fibrosis (IPF), familial interstitial pulmonary fibrosis and asbestosis.

In a further particular preferred embodiment, the present invention provides a pharmaceutical combination described herein for use in a method of preventing or treating lung fibrosis, wherein the lung fibrosis is selected from the group consisting of idiopathic pulmonary fibrosis (IPF), familial interstitial pulmonary fibrosis, chronic obstructive pulmonary disease (COPD), and asbestosis.

In a more particular preferred embodiment, the present invention provides a pharmaceutical combination described herein for use in a method of preventing or treating idiopathic pulmonary fibrosis (IPF).

In one embodiment, the present invention provides a pharmaceutical combination comprising:

(a) a PPAR agonist;

(b) a p38 kinase inhibitor; and optionally

(c) one or more pharmaceutically acceptable diluents, excipients or carriers for use in a method of preventing or treating fibrotic diseases or disorders in a subject, wherein said p38 kinase inhibitor is preferably inhibiting p38-alpha, p38-beta, p38-gamma or p38-delta or combinations thereof; more preferably inhibiting p38-alpha and/or p38-beta.

In one embodiment, the present invention provides a pharmaceutical combination comprising:

(a) a PPAR agonist;

(b) a p38 kinase inhibitor; and optionally

(c) one or more pharmaceutically acceptable diluents, excipients or carriers for use in a method of preventing or treating fibrotic diseases or disorders in a subject, wherein said p38 kinase inhibitor is selected from the group consisting of pamapimod, acumapimod, losmapimod, dilmapimod, semapimod, AZD7624, ARRY-371797, LY2228820, R9111, PH-797804, BIRB 796, VX-702, VX-745 SB 239063, SB202190, SCIO 469, and BMS 582949 or a pharmaceutically acceptable salt thereof.

In a further embodiment, the present invention provides a pharmaceutical combination according to the invention, comprising

(a) a PPAR agonist;

(b) a p38 kinase inhibitor, wherein said p38 kinase inhibitor is a compound of formula I or a pharmaceutically acceptable salt thereof as defined herein; and optionally

(c) one or more pharmaceutically acceptable diluents, excipients or carriers for use in a method of preventing or treating fibrotic diseases or disorders in a subject.

In a further embodiment, the present invention provides a pharmaceutical combination comprising

(a) a PPAR agonist;

(b) a p38 kinase inhibitor, wherein said p38 kinase inhibitor is a compound of formula II or a pharmaceutically acceptable salt thereof as defined herein; and optionally

(c) one or more pharmaceutically acceptable diluents, excipients or carriers for use in a method of preventing or treating fibrotic diseases or disorders in a subject.

In a preferred embodiment, the present invention provides a pharmaceutical combination comprising:

(a) a PPAR agonist;

(b) a p38 kinase inhibitor, wherein said p38 kinase inhibitor is pamapimod or a pharmaceutically acceptable salt thereof; and optionally

(c) one or more pharmaceutically acceptable diluents, excipients or carriers for use in a method of preventing or treating fibrotic diseases or disorders in a subject.

In a further preferred embodiment, the present invention provides a pharmaceutical combination comprising:

(a) a PPAR agonist;

(b) a p38 kinase inhibitor, wherein said p38 kinase inhibitor is a compound of formula I or a pharmaceutically acceptable salt thereof as defined herein; and optionally

(c) one or more pharmaceutically acceptable diluents, excipients or carriers for use in a method of preventing or treating fibrotic diseases or disorders in a subject, wherein the PPAR gamma agonist is selected from the group consisting of pioglitazone, rosiglitazone and troglitazone or pharmaceutically acceptable salts thereof.

In a further preferred embodiment, the present invention provides a pharmaceutical combination comprising:

(d) a PPAR agonist;

(e) a p38 kinase inhibitor, wherein said p38 kinase inhibitor is pamapimod or a pharmaceutically acceptable salt thereof; and optionally

(f) one or more pharmaceutically acceptable diluents, excipients or carriers for use in a method of preventing or treating fibrotic diseases or disorders in a subject, wherein the PPAR gamma agonist is selected from the group consisting of pioglitazone, rosiglitazone and troglitazone or pharmaceutically acceptable salts thereof.

In a further embodiment, the present invention provides a pharmaceutical combination comprising

(a) a PPAR gamma agonist;

(b) a p38 kinase inhibitor, wherein said p38 kinase inhibitor is a compound of formula I or a pharmaceutically acceptable salt thereof as defined herein; and optionally

(c) one or more pharmaceutically acceptable diluents, excipients or carriers for use in a method of preventing or treating fibrotic diseases or disorders in a subject.

In a further embodiment, the present invention provides a pharmaceutical combination comprising

(a) a PPAR gamma agonist;

(b) a p38 kinase inhibitor, wherein said p38 kinase inhibitor is a compound of formula II or a pharmaceutically acceptable salt thereof as defined herein; and optionally

(c) one or more pharmaceutically acceptable diluents, excipients or carriers for use in a method of preventing or treating fibrotic diseases or disorders in a subject.

In a preferred embodiment, the present invention provides a pharmaceutical combination comprising

(a) a PPAR gamma agonist;

(b) a p38 kinase inhibitor, wherein said p38 kinase inhibitor is a compound of formula I or a pharmaceutically acceptable salt thereof as defined herein; and optionally

(c) one or more pharmaceutically acceptable diluents, excipients or carriers for use in a method of preventing or treating fibrotic diseases or disorders in a subject;

wherein said PPAR gamma agonist is selected from the group consisting of pioglitazone, rosiglitazone, troglitazone and INT131 or a pharmaceutically acceptable salt thereof, preferably selected from the group consisting of pioglitazone, rosiglitazone and troglitazone or a pharmaceutically acceptable salt thereof; and

wherein X¹ and X² in said compound of formula I are each O; and

wherein Z in said compound of formula I is N; and

wherein W in said compound of formula I is NH; and

wherein Ar¹ in said compound of formula I is aryl; and

wherein R¹ in said compound of formula I is heteroalkyl; and

wherein R³ in said compound of formula I is alkyl.

In a further preferred embodiment, the present invention provides a pharmaceutical combination comprising

(a) a PPAR gamma agonist;

(b) a p38 kinase inhibitor, wherein said p38 kinase inhibitor is pamapimod, R9111, semapimod, or a pharmaceutically acceptable salt thereof, preferably pamapimod or a pharmaceutically acceptable salt thereof; and optionally

(c) one or more pharmaceutically acceptable diluents, excipients or carriers for use in a method of preventing or treating fibrotic diseases or disorders in a subject; wherein said PPAR gamma agonist is selected from the group consisting of pioglitazone, troglitazone, bezafibrate and pharmaceutically acceptable salts thereof.

In a further preferred embodiment, the present invention provides a pharmaceutical combination comprising

(a) a PPAR gamma agonist;

(b) a p38 kinase inhibitor, wherein said p38 kinase inhibitor is pamapimod, losmapimod, dilmapimod or R9111, or a pharmaceutically acceptable salt thereof, preferably pamapimod or dilmapimod or a pharmaceutically acceptable salt thereof, more preferably pamapimod or a pharmaceutically acceptable salt thereof; and optionally

(c) one or more pharmaceutically acceptable diluents, excipients or carriers for use in a method of preventing or treating fibrotic diseases or disorders in a subject.

In a further preferred embodiment, the present invention provides a pharmaceutical combination comprising

(d) a PPAR gamma agonist;

(e) a p38 kinase inhibitor, wherein said p38 kinase inhibitor is pamapimod, losmapimod, dilmapimod or R9111, or a pharmaceutically acceptable salt thereof, preferably pamapimod or dilmapimod or a pharmaceutically acceptable salt thereof, more preferably pamapimod or a pharmaceutically acceptable salt thereof; and optionally

(f) one or more pharmaceutically acceptable diluents, excipients or carriers for use in a method of preventing or treating fibrotic diseases or disorders in a subject;

wherein said PPAR gamma agonist is selected from the group consisting of pioglitazone, rosiglitazone and troglitazone or pharmaceutically acceptable salts thereof.

In one embodiment, the present invention provides a pharmaceutical combination comprising:

(a) a PPAR agonist;

(b) a p38 kinase inhibitor; and optionally

(c) one or more pharmaceutically acceptable diluents, excipients or carriers for use in a method of preventing or treating fibrotic diseases or disorders in a subject, wherein said PPAR agonist is activating PPAR alpha, PPAR gamma or PPAR delta or combinations thereof.

In one embodiment, the present invention provides a pharmaceutical combination comprising:

(a) a PPAR agonist;

(b) a p38 kinase inhibitor; and optionally

(c) one or more pharmaceutically acceptable diluents, excipients or carriers for use in a method of preventing or treating fibrotic diseases or disorders in a subject, wherein said PPAR agonist is selected from the group consisting of pioglitazone, troglitazone, rosiglitazone, bezafibrate, fenofibrate, clofibrate, gemfibrozil, aleglitazar, muraglitazar, tesaglitazar, ragaglitazar, saroglitazar, GFT505, naveglitazar, GW501516 and INT131 or a pharmaceutically acceptable salt thereof.

In one embodiment, the present invention provides a pharmaceutical combination comprising:

(a) a PPAR agonist;

(b) a p38 kinase inhibitor; and optionally

(c) one or more pharmaceutically acceptable diluents, excipients or carriers for use in a method of preventing or treating fibrotic diseases or disorders in a subject, wherein said PPAR agonist is selected from the group consisting of pioglitazone, troglitazone, bezafibrate and pharmaceutically acceptable salts thereof.

In a preferred embodiment, the present invention provides a pharmaceutical combination comprising:

(a) a PPAR agonist;

(b) a p38 kinase inhibitor; and optionally

(c) one or more pharmaceutically acceptable diluents, excipients or carriers for use in a method of preventing or treating fibrotic diseases or disorders in a subject, wherein said PPAR agonist is pioglitazone or a pharmaceutically acceptable salt thereof.

In a preferred embodiment, the present invention provides a pharmaceutical combination comprising:

(a) a PPAR agonist;

(b) a p38 kinase inhibitor; and optionally

(c) one or more pharmaceutically acceptable diluents, excipients or carriers for use in a method of preventing or treating fibrotic diseases or disorders in a subject, wherein said PPAR agonist is selected from the group consisting of pioglitazone, rosiglitazone and troglitazone or pharmaceutically acceptable salts thereof.

In a further preferred embodiment, the present invention provides a pharmaceutical combination comprising:

(a) a PPAR agonist, wherein said PPAR agonist is pioglitazone or a pharmaceutically acceptable salt thereof;

(b) a p38 kinase inhibitor; and optionally

(c) one or more pharmaceutically acceptable diluents, excipients or carriers for use in a method of preventing or treating fibrotic diseases or disorders in a subject.

In a further preferred embodiment, the present invention provides a pharmaceutical combination comprising:

(a) a PPAR agonist, wherein said PPAR agonist is pioglitazone hydrochloride;

(b) a p38 kinase inhibitor; and optionally

(c) one or more pharmaceutically acceptable diluents, excipients or carriers for use in a method of preventing or treating fibrotic diseases or disorders in a subject.

In a particularly preferred embodiment, the present invention provides a pharmaceutical combination comprising:

(a) a PPAR agonist, wherein said PPAR agonist is pioglitazone or a pharmaceutically acceptable salt thereof, preferably pioglitazone hydrochloride;

(b) a p38 kinase inhibitor, wherein said p38 kinase inhibitor is pamapimod or a pharmaceutically acceptable salt thereof; and optionally

(c) one or more pharmaceutically acceptable diluents, excipients or carriers for use in a method of preventing or treating fibrotic diseases or disorders in a subject.

In a further particularly preferred embodiment, the present invention provides a pharmaceutical combination comprising:

(a) a PPAR agonist, wherein said PPAR agonist is pioglitazone or a pharmaceutically acceptable salt thereof, preferably pioglitazone hydrochloride;

(b) a p38 kinase inhibitor, wherein said p38 kinase inhibitor is pamapimod or a pharmaceutically acceptable salt thereof; and optionally

(c) one or more pharmaceutically acceptable diluents, excipients or carriers for use in a method of preventing or treating fibrotic diseases or disorders in a subject, wherein said fibrotic diseases or disorders is idiopathic pulmonary fibrosis (IPF).

### EXAMPLES

The present Examples are intended to illustrate the present invention without restricting it.

### Example 1: Combination Treatment with Pamapimod and Pioglitazone Protects Against Bleomycin-Induced Lung Fibrosis

1. Summary

Bleomycin is widely used to induce pulmonary fibrosis in mice in order to study potential therapies. The efficacy of pioglitazone and pamapimod, dosed individually, and in combination, in comparison to the approved drug pirfenidone, were tested in a 21-day model of bleomycin-induced pulmonary fibrosis in mice. Both once daily pioglitazone and once daily pamapimod, dosed individually and in combination, significantly reduced normalized lung weights and fibrosis scores compared to vehicle-treated controls. The combination of pamapimod/pioglitazone group showed greater reductions in these disease paramenters than the groups receiving the single agents. All treatment groups showed greater efficacy than the positive control group receiving twice daily pirfenidone. These results show that the combination of pioglitazone and pamapimod is effective to reduce bleomycin-induced lung fibrosis and is a candidate therapeutic regimen for the treatment of lung fibrosis.

2. Materials and Methods

2.1. Study Animals

Male C57BL/6 mice, six to seven weeks of age, were used for this study. Animals were weighed one day prior to study initiation and 60 animals were randomized into 6 groups (N=10/group) such that mean body weights were similar for the different groups. Food and water were provided ad libitum, with a 12-hour light/dark cycle.

2.2. Study Design

Treatment in groups 2-6 was initiated one day prior to bleomycin administration and continued though to the study end on day 21. On day -1, animals were dosed via oral gavage with vehicle control (0.5% methylcellulose; group 1 and group 2), pioglitazone at 25 mg/kg/dose (group 3), pamapimod at 100 mg/kg/dose (group 4), pioglitazone at 25 mg/kg/dose and pamapimod at 100 mg/kg/dose (group 5), pirfenidone at 100mg/kg/dose (group 6) and continued for 21 days. Pioglitazone and pamapimod were dosed once daily (QD) and pirfenidone was dosed twice daily (BID). On day 0, all mice in groups 2-6 received a single instillation of bleomycin to induce pulmonary fibrosis. Animals in group 1 (dosed with vehicle from days-1 to 21) were not instilled with bleomycin, but instead received a single dose of saline, and were considered sham negative control mice. Final drug treatments were administered 2-4 hours prior to study termination on day 21.

Body weights, absolute lung weights, and lung weights normalized to body weights were measured for all mice on day 21 after bleomycin instillation, and were compared to bleomycin-instilled vehicle treated control mice. The post-caval lobes from the right lung were collected and snap frozen. The remaining lungs were fixed in 10% NBF and underwent histopathology analysis and evaluated for eseverity of fibrosis using a modified Ashcroft score.

2.3. Compounds and Vehicle Formulation

Vehicle (0.5% methylcellulose) and compounds were prepared weekly and stored at room temperature in the dark. All mice were dosed starting on day-1 until study termination on day 21 with a volume of 200 μl of vehicle or test compounds QD by oral gavage or 100 μl of pirfenidone BID by oral gavage. For groups receiving both pirfenidone and test compounds, pirfenidone was dosed 100 μl BID and test compounds were delivered in a total volume of 100 μl QD.

2.4. Harvest Procedures

This study was terminated on day 21 for all groups approximately 2 to 4 hours after final compound or vehicle control treatment. All mice were anesthetized with isoflurane inhalant anesthesia and sacrificed by cervical dislocation.

The lungs from each animal were harvested and weighed. The post-caval lobe from the right lung was separated by tying a suture so that there was no leakage from the rest of the lung. The post caval lobe was weighed and snap frozen for further analysis. The remaining lungs from each animal were inflated with 10% neutral buffered formalin (NBF) and then fixed in 10% NBF for histology.

2.5. Fibrosis Scoring

The lung samples were processed and embedded with all lobes from each mouse in one paraffin block. Coronal sections through the four major lobes were stained with Masson's Trichrome. For each animal, consecutive lung fields were examined in a raster pattern using a 20× objective lens and a 10× ocular lens (200×). A modified Ashcroft score (Hubner et al., 2008) was recorded for each field. The fibrotic index was calculated as the sum of the modified Ashcroft field scores divided by the number of fields examined. Results of the histopathology microscopic evaluation were entered directly into Excel 2016 spreadsheets.

Endpoint data on day 21 is shown as the average Ashcroft score per experimental group, with standard error of the mean (SEM). Statistical differences (defined as p<0.05) between groups were analyzed using t-tests in Prism version 7.0 software (GraphPad, La Jolla, Calif.). Groups treated with test compounds were compared to bleomycin-instilled vehicle control treated mice.

3. Results

3.1. Absolute and Normalized Lung Weights

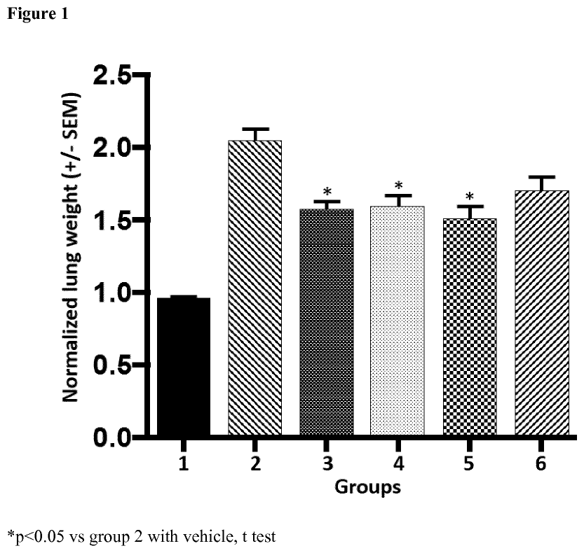

As shown by FIG. 1, bleomycin instillation led to a significant increase in normalized lung weight when compared to non-bleomycin instilled sham controls. Substantially lower normalized lung weights were observed in groups treated with 25 mg/kg pioglitazone or 100 mg/kg pamapimod either alone or in combination (Groups 3, 4, and 5 vs. group 2, p<0.05, t test). Notably the combination reduced normalized lung weights to a greater extent than the positive control pirfenidone (Group 6).

3.2. Fibrosis Score

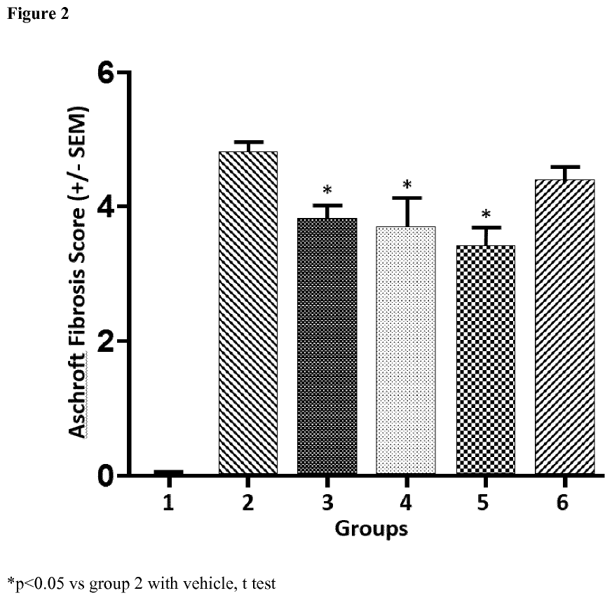

As shown by FIG. 2, induction with 1.5 U/kg bleomycin followed by 200 μL vehicle daily (Group 2) resulted in substantial fibrosis and the highest group mean fibrotic index (4.8).

Substantially less fibrosis, as indicated by lower group mean fibrotic indices, was present in groups treated with 25 mg/kg pioglitazone or 100 mg/kg pamapimod either alone or in combination (Groups 3, 4, and 5 vs. group 2, p<0.05, t test). The lowest group mean fibrotic index was in Group 5, indicating that the combination of pioglitazone and pamapimod is more effective to reduce fibrosis score than either agent alone. Notably the combination reduced normalized fibrosis score to a greater extent than the positive control pirfenidone (Group 6).

Example 2: Combination Treatment with Pamapimod and Pioglitazone Shows Synergy on Interleukin and TNF Pathway Gene Expression Changes in Mouse Lung Tissue

1. Summary

Fibrosis is a pathological process characterized by the replacement of normal tissue by mesenchymal cells and extracellular matrix. The sequence of events leading to fibrosis of the lung involves inflammation and disruption of normal tissue architecture. Several cytokines participate in local injury and inflammation. These include interleukin-1 (IL-1), interleukin-8 (IL-8), Interleukin-6 as well as tumor necrosis factor-alpha (TNF-alpha)] and related molecules. We performed whole genome expression analysis on lung tissue from the bleomycin fibrosis study presented in Example 1. Surprisingly, the results revealed that pioglitazone and pamapimod have a synergistic effect to significantly regulate the expression of multiple interleukin/interleukin receptor as well as TNF/TNF receptor genes. These data suggest that the combination of the two drugs may provide better therapeutic efficacy in the treatment of lung fibrosis than either drug alone.

2. Materials and Methods

Global gene expression changes were determined by Illumina Next Generation RNA sequencing in lung samples from the animal study described in Example 1. RNA was extracted from the post caval lobe that had been snap frozen at the termination of the study. For this analysis, 8 samples from each of treatment groups 1-6 (total 48 samples) were analysed. Briefly, total RNA was extracted for next generation sequencing using standard methods, then Illumina TruSeq RNA libraries including poly(A) enrichment were prepared. Sequencing was performed on an Illumina NextSeq 500, v2, high output, 1×75 bp reads with 30 Mio packages. Demultiplexing and trimming of Illumina adaptor residuals was performed on the raw data. Mapping of data reads was made using the reference mouse genome mm10. For bioinformatics analysis, the average expression level and standard deviations were determined for each gene for the 8 replicates per treatment group. Pairwise comparisons between groups were then performed to identify differentially expressed genes. Absolute p-values and adjusted p-values, to correct for multiplicity of testing, were calculated for each pairwise analysis. Data output consisted of the top 1000 differentially expressed genes. A gene was considered significantly up or downregulated if the difference in a between-group comparison yielded an adjusted p value <0.10.

3. Results

Table 1 below shows the significantly regulated genes (adjusted p value <0.10) for the three treatment groups in comparison to the the bleomycin alone disease group. Neither pioglitazone or pamapimod single treatments significantly regulated expression of members of the interleukin/interleukin receptor, TNF/TNF receptor, or C-C and C-X-C motif chemokine families. Surprisingly, combined treatment with pioglitazone/pamapimod significantly repressed a very large number of inflammatory cytokines belonging to these four families. Many of these have been implicated in the pathogenesis of IPF. These data strongly support synergy of the combination, indicating that the combination has potentially potent anti-inflammatory effects, not exhibited by either agent alone.

TABLE 1
Synergistic effect of the pamapimod/pioglitazone combination on cytokines and chemokines
of the interleukin, TNF, C-C and C-X-C motif families in the mouse bleomycin model
RNASeq whole genome lung expression data from Mouse bleomycin study 2

| | | adjusted significance (p) value | | | |
|---|---|---|---|---|---|
| Gene | Gene ID | Pioglitazone | Pamapimod | Combination | Direction |
| Interleukins and receptors | | | | | |
| Interleukin 6 | IL6 | NS | NS | 0.000161 | downregulated |
| Interleukin 12B | IL12b | NS | 0.023 | 0.0142 | downregulated |
| Interleukin 36 Gamma | IL1f9 | NS | NS | 0.033 | upregulated |
| Interleukin 11 | IL11 | NS | NS | 0.054 | downregulated |
| Interleukin 18 Receptor 1 | IL18r1 | NS | NS | 0.076 | downregulated |
| Interleukin 1 Receptor Type 2 | IL1r2 | NS | NS | 0.084 | upregulated |
| TNFs and receptors | | | | | |
| TNF Receptor Superfamily Member 11b | Tnfrsf11b | NS | NS | 0.0084 | downregulated |
| TNF Superfamily Member 9 | Tnfsf9 | NS | NS | 0.028 | downregulated |
| TNF Receptor Superfamily Member 10b | Tnfrsf10b | NS | NS | 0.055 | downregulated |
| TNF Superfamily Member 18 | Tnfsf18 | NS | NS | 0.073 | downregulated |
| TNF Superfamily Member 15 | Tnfsf15 | NS | 0.0079 | NS | upregulated |
| Proinflammatory chemokines | | | | | |
| C-C Motif Chemokine Ligand 7 | Ccl7 | NS | NS | 0.0014 | downregulated |
| C-C Motif Chemokine Ligand 4 | Ccl4 | NS | 0.042 | 0.0032 | downregulated |
| C-C Motif Chemokine Ligand 3 | Ccl3 | NS | NS | 0.0048 | downregulated |
| C-C Motif Chemokine Ligand 12 | Ccl12 | NS | NS | 0.021 | downregulated |
| C-C Motif Chemokine Ligand 2 | Ccl2 | NS | NS | 0.023 | downregulated |
| C-C Motif Chemokine Ligand 8 | Ccl8 | NS | NS | 0.08 | downregulated |
| C-C Motif Chemokine Ligand 24 | Ccl24 | NS | 0.032 | NS | downregulated |
| C-X-C Motif Chemokine Ligand 10 | Cxcl10 | NS | NS | 0.048 | downregulated |
| C-X-C Motif Chemokine Ligand 5 | Cxcl5 | NS | NS | 0.052 | downregulated |
| C-X-C Motif Chemokine Ligand 3 | Cxcl3 | NS | NS | 0.094 | downregulated |

NS = adjusted p value > 0.10

1. A pharmaceutical combination comprising:
(a) a PPAR agonist;
(b) a p38 kinase inhibitor; and optionally
(c) one or more pharmaceutically acceptable diluents, excipients or carriers for use in a method of preventing or treating fibrotic diseases or disorders in a subject.

2. The pharmaceutical combination for use according to claim 1, wherein said p38 kinase inhibitor is inhibiting p38-alpha and/or p38-beta.

3. The pharmaceutical combination for use according to claim 1, wherein said p38 inhibitor is a compound of formula I or II 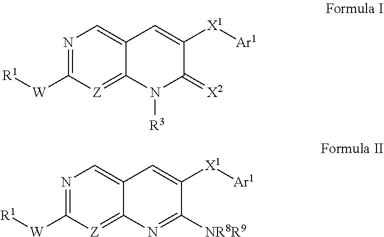
or a pharmaceutically acceptable salt thereof, wherein
Z is N or CH;
W is NR²;
X¹ is O, NR⁴ (where R⁴ is hydrogen or alkyl), S, or CR⁵R⁶ (where R⁵ and R⁶ are independently hydrogen or alkyl) or C═O;
X² is O or NR⁷;
Ar¹ is aryl or heteroaryl;
R² is hydrogen, alkyl, acyl, alkoxycarbonyl, aryloxycarbonyl, heteroalkylcarbonyl, heteroalkyloxycarbonyl or —R²¹—R22 where R²¹ is alkylene or —C(═O)— and R²² is alkyl or alkoxy;
R¹ is hydrogen, alkyl, haloalkyl, aryl, aralkyl, heteroaryl, heteroaralkyl, cycloalkyl, cycloalkylalkyl, heteroalkylsubstituted cycloalkyl, heterosubstituted cycloalkyl, heteroalkyl, cyanoalkyl, heterocyclyl, heterocyclylalkyl, R¹²—SO₂-heterocycloamino (where R¹² is haloalkyl, aryl, aralkyl, heteroaryl or heteroaralkyl), —Y¹—C(O)—Y²—R¹¹ (where Y¹ and Y² are independently either absent or an alkylene group and R¹¹ is hydrogen, alkyl, halo alkyl, hydroxy, alkoxy, amino, monoalkylamino or dialkylamino), (heterocyclyl)(cycloalkyl)alkyl or (heterocyclyl)(heteroaryl)alkyl;
R³ is hydrogen, alkyl, cycloalkyl, cycloalkylalkyl, aryl, aralkyl, haloalkyl, heteroalkyl, cyanoalkyl, alkylene-C(O)—R³¹ (where R³¹ is hydrogen, alkyl, hydroxy, alkoxy, amino, monoalkylamino or dialkylamino), amino, monoalkylamino, dialkylamino or NR³²—Y³—R³³ (where Y³ is —C(O), —C(O)O—, —C(O)NR³⁴, S(O)₂ or S(O)₂NR³⁵; R³², R³⁴ and R³⁵ are independently hydrogen or alkyl; and R³³ is hydrogen, alkyl, cycloalkyl, cycloalkylalkyl, heteroalkyl or optionally substituted phenyl) or acyl;
R⁷ is hydrogen or alkyl; and
R⁸ and R⁹ are independently hydrogen, alkyl, aryl, aralkyl, cycloalkyl, cycloalkylalkyl, heteroalkyl, alkylsulfonyl, arylsulfonyl, —C(O)—R⁸¹ (where R⁸¹ is alkyl, aryl, aralkyl, cycloalkyl, cycloalkylalkyl, heteroalkyl, alkoxy, aryloxy, amino, mono- or di-alkylamino, arylamino or aryl(alkyl)amino) or R⁸ and R⁹ together form ═CR⁸²R⁸³ (where R⁸² and R⁸³ are independently hydrogen, alkyl, cycloalkyl, cycloalkylalkyl or optionally substituted phenyl) and optionally one or more pharmaceutically acceptable diluents, excipients or carriers.

4. The pharmaceutical combination for use according to claim 3, wherein said p38 inhibitor is a compound of formula I or a pharmaceutically acceptable salt thereof 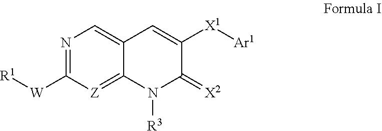
wherein Ar¹, W, X¹, X², Z, R¹ and R³ are as defined in claim 3.

5. The pharmaceutical combination for use according to claim 1, wherein said p38 kinase inhibitor is selected from the group consisting of pamapimod, acumapimod, losmapimod, dilmapimod, semapimod, AZD7624, ARRY-371797, LY2228820, R9111, PH-797804, BIRB 796, VX-702, VX-745, SB 239063, SB202190, SCIO 469, BMS 582949 and pharmaceutically acceptable salts thereof.

6. The pharmaceutical combination for use according to claim 1, wherein said p38 kinase inhibitor is pamapimod (6-(2,4-Difluorophenoxy)-2-[3 -hydroxy-1-(2-hydroxyethyl)-propylamino]-8-methyl-8H-pyrido[2,3-d]pyrimidin-7-one, Formula III) or a pharmaceutically acceptable salt thereof.

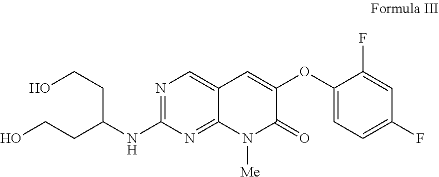 [SMILES: Cn1c(=O)c(Oc2ccc(F)cc2F)cc2cnc(NC(CCO)CCO)nc21]

7. The pharmaceutical combination for use according to any one of claims 1-6, wherein said PPAR agonist is activating PPAR gamma and/or PPAR alpha.

8. The pharmaceutical combination for use according to any one of claims 1-6, wherein said PPAR agonist is activating PPAR gamma.

9. The pharmaceutical combination for use according to any one of claims 1-6, wherein said PPAR agonist is selected from the group consisting of pioglitazone, rosiglitazone troglitazone, fenofibrate, bezafibrate and pharmaceutically acceptable salts thereof.

10. The pharmaceutical combination for use according to any one of claims 1-6, wherein said PPAR agonist is pioglitazone or a pharmaceutically acceptable salt thereof.

11. The pharmaceutical combination for use according to any one of claims 1-10, wherein said combination is administered orally to the subject.

12. The pharmaceutical combination for use according to any one of claims 1-11, wherein said fibrotic disease or disorder is selected from the group consisting of lung fibrosis, liver fibrosis, kidney fibrosis, cardiac fibrosis, ocular fibrosis or cutaneous fibrosis.

13. The pharmaceutical combination for use according to any one of claims 1-11, wherein said fibrotic disease or disorder is lung fibrosis.

14. The pharmaceutical combination for use according to claim 13, wherein said lung fibrosis is selected from the group consisting of idiopathic pulmonary fibrosis (IPF), familial interstitial pulmonary fibrosis, nonspecific interstitial pneumonia (NSIP), cryptogenic organizing pneumonia (COP), sarcoidosis, chronic obstructive pulmonary disease (COPD), and asbestosis.

15. A kit for use in a method of preventing or treating fibrotic diseases or disorders in a subject, comprising a pharmaceutical combination comprising:
(a) a PPAR agonist;
(b) a p38 kinase inhibitor; and optionally
(c) one or more pharmaceutically acceptable diluents, excipients or carriers; and instructions for using the kit.
